(19) United States
(12) Patent Application Publication
(10) Pub. No.: US 2025/0228714 A1
(43) Pub. Date: Jul. 17, 2025

(54) ABSORBENT ARTICLES AND METHODS FOR MAKING ABSORBENT ARTICLES WITH A WAIST PANEL AND A POCKET POSITIONED BETWEEN A TOPSHEET AND A BACKSHEET

(71) Applicant: The Procter & Gamble Company, Cincinnati, OH (US)

(72) Inventors: Uwe SCHNEIDER, Cincinnati, OH (US); Jeromy Thomas RAYCHECK, South Lebanon, OH (US); Abhishek SURUSHE, Mason, OH (US)

(21) Appl. No.: 19/013,012

(22) Filed: Jan. 8, 2025

Related U.S. Application Data
(60) Provisional application No. 63/621,667, filed on Jan. 17, 2024.

(51) Int. Cl. A61F 13/15; A61F 13/49

(52) U.S. Cl. CPC A61F 13/15739; A61F 13/49011

(57) ABSTRACT

The present disclosure relates to absorbent articles with a waist panel and a pocket positioned between a topsheet and a backsheet. The waist panel is bonded with a garment facing surface of the topsheet. A first region of a second surface the waist panel is bonded with a wearer facing surface of the backsheet, and a second region is unattached to the backsheet to define a pocket between the second region and the backsheet. The pocket comprises an opening defined by a slit in the topsheet. In an assembly method, a slit is cut into a first carrier substrate. An elastic part is sandwiched between the first carrier substrate and a second carrier substrate. A first region of the elastic part is bonded with the second carrier substrate, and a second region remains unattached to the second carrier substrate to define a pocket with an opening defined by the slit.

### CROSS REFERENCE TO RELATED APPLICATION

This application claims the benefit of U.S. Provisional Application No. 63/621,667, filed Jan. 17, 2024, which is incorporated by reference herein in its entirety.

### FIELD

The present disclosure relates to absorbent articles including waist panels, and more particularly, absorbent articles and methods for making absorbent articles with a waist panel and a pocket positioned between a topsheet and a backsheet.

### BACKGROUND

Along an assembly line, various types of articles, such as for example, diapers and other absorbent articles, may be assembled by adding components to and/or otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. In some cases, individual components created from an advancing web or webs are combined with other individual components created from other advancing webs. Webs of material and component parts used to manufacture diapers may include: backsheets, topsheets, leg cuffs, waistbands, absorbent core components, front and/or back ears, fastening components, and various types of elastic webs and parts such as leg elastics, barrier leg cuff elastics, stretch side panels, and waist elastics. Once the desired component parts are assembled, the advancing web(s) and component parts are subjected to a final knife cut to separate the web(s) into discrete diapers or other absorbent articles.

Some absorbent articles, such as diapers, have components that include waist panels, which may also be referred to as waistbands. In some configurations, waistbands may be provided as a single layer of elastic material, such as an elastic film. In some configurations, the waistbands may be provided as an elastic laminate that may include elastic material bonded to one or more substrates such as nonwovens, wherein the elastic material may include an elastic film and/or elastic strands. In some assembly operations, the waistbands are joined to an advancing carrier web, such as a continuous topsheet or backsheet web, while the waistbands are in a stretched condition. As such, when the waistbands relax, the carrier web gathers to form corrugations. The resulting laminate is stretchable to the extent that the corrugations allow the waistband to elongate.

When manufacturing diapers, the waistband may be provided as a continuous length of waistband material that may be stretched; cut into discrete waistbands; and bonded with the advancing carrier web, such as a continuous topsheet or backsheet web, while the waistband is in a stretched state. With some diapers, it may be desirable to include a front waistband in a front waist region and a back waistband in an opposing back waist region. Some assembly operations may apply a piece of waistband material to the advancing carrier web that is subsequently cut into separate front and back waistbands when the advancing carrier web is subject to the final knife cut that separates the carrier web into discrete diapers. In turn, the front and back waistbands may be created from the same continuous length of waistband material.

In some configurations, it may be desirable to provide diapers with front and/or rear waistbands having regions that are bonded with a topsheet, and one or more regions that are not bonded (unattached) with the topsheet, thereby forming a pocket between the waistband and the topsheet. Such a pocket may help contain bodily exudates and/or help prevent leaks from an absorbent article. In some configurations, the pocket may be formed by attaching a waistband to a topsheet along three edges of the waistband wherein a fourth edge of the waistband remains unattached to the topsheet.

However, assembling absorbent articles with waistbands having unattached edges may create certain production challenges, which may be exacerbated at the high speed production rates of some absorbent article processes. For example, depending on a particular manufacturing configuration, the unattached edge of a waistband may be oriented so as define a leading edge of the waistband while advancing through various assembly operations. As such, in some instances, air may be forced into the pocket while the waistband is advancing through assembly operations, causing the waistband to act like a sail or parachute by repeatedly lifting and/or separating from the topsheet and/or otherwise deforming the waistband shape. Such repeated lifting and/or deformation may cause the waistband to be partially or whole torn away from the topsheet. In some instances, the unattached edge of the waistband may be forced into areas of the topsheet with freshly applied adhesive between the topsheet and the waistband, causing a portion of the unattached edge to be unintentionally bonded with topsheet. In some configurations, frictional forces acting on diaper components advancing along various assembly apparatuses, such as folding plows, may drag or pull on the unattached edge, in turn, causing permanent deformation and/or removal of a portion or an entirety of the waistband.

Consequently, it would be beneficial to provide diapers with waist panels configured with an edge that is unattached from other diaper components so as to form a pocket during use, and wherein a region of the waist panel may be positioned relative to other diaper components, so as to help prevent and/or reduce instances of separation of the waist panel from such absorbent article components during assembly.

### SUMMARY

In one form, a method for assembling absorbent articles comprises steps of: providing a first carrier substrate; advancing the first carrier substrate in a machine direction; providing an elastic part comprising a first surface and an opposing second surface, the elastic part further comprising a first longitudinal edge and a second longitudinal edge separated from the first longitudinal edge in a cross direction, the elastic part further comprising a first lateral edge and a second lateral edge separated from the first lateral edge in the machine direction; bonding the first surface of the elastic part with the first carrier substrate; cutting a slit in the first carrier substrate, the slit extending in the cross direction; providing a second carrier substrate; sandwiching the elastic part between the first carrier substrate and the second carrier substrate; and bonding a first region of the second surface the elastic part with the second carrier substrate, wherein a second region of the second surface of the elastic part remains unattached to the second carrier substrate to define a pocket between the second region of the second surface of the elastic part and the second carrier substrate, the pocket comprising an opening defined by the slit in the first carrier substrate.

In another form, an absorbent article comprises: a front waist region, a back waist region, and a crotch region disposed between the front and back waist regions; a first waist edge, a second waist edge longitudinally separated from the first waist edge, a first side edge; and a second side edge laterally separated from the first side edge; a chassis comprising a topsheet, a backsheet, and an absorbent core positioned between the topsheet and the backsheet; a slit in the topsheet; a waist panel comprising an inboard lateral edge, an outboard lateral edge, a first longitudinal edge, and a second longitudinal edge, wherein the waist panel further comprises a first surface and an opposing second surface; wherein the first surface of the waist panel is bonded with a garment facing surface of the topsheet, and wherein a first region of the second surface the waist panel is bonded with a wearer facing surface of the backsheet, wherein a second region of the second surface of the waist panel is unattached to the backsheet to define a pocket between the second region of the second surface of the waist panel and the backsheet, the pocket comprising an opening defined by the slit in the topsheet.

### BRIEF DESCRIPTION OF THE DRAWINGS

FIG. 1A is a partially cut away plan view of an absorbent article in the form of a taped diaper that may include one or more substrates assembled in accordance with the present disclosure with the portion of the diaper that faces away from a wearer oriented towards the viewer.

FIG. 1B is a plan view of the absorbent article of FIG. 1A that may include one or more substrates assembled in accordance with the present disclosure with the portion of the diaper that faces toward a wearer oriented towards the viewer.

FIG. 1C is a plan view of an absorbent article that may include one or more substrates assembled in accordance with the present disclosure with the portion of the diaper that faces toward a wearer oriented towards the viewer.

FIG. 2 is a detailed view of a first waist panel positioned between a topsheet and a backsheet with the portion of the diaper that faces toward a wearer oriented towards the viewer.

FIG. 2A1 is a detailed view of the first waist panel from FIG. 2 illustrating a first bonding configuration between a first surface of the waist panel and the topsheet.

FIG. 2A2 is a detailed view of the first waist panel from FIG. 2 illustrating a first bonding configuration between a second surface of the waist panel and the backsheet.

FIG. 2B1 is a detailed view of the first waist panel from FIG. 2 illustrating a second bonding configuration between a first surface of the waist panel and the topsheet.

FIG. 2B2 is a detailed view of the first waist panel from FIG. 2 illustrating a second bonding configuration between a second surface of the waist panel and the backsheet.

FIG. 2C1 is a detailed view of the first waist panel wherein the slit extends through the waist panel and illustrating a third bonding configuration between a first surface of the waist panel and the topsheet.

FIG. 2C2 is a detailed view of the first waist panel wherein the slit extends through the waist panel and illustrating a second bonding configuration between a second surface of the waist panel and the backsheet.

FIG. 3 is a schematic side view of an apparatus for bonding elastic parts to advancing first and second carrier webs.

FIG. 4 is a view of a cutting device, transfer device, and bonding device taken along section 4-4 in FIG. 3.

FIG. 5 is a view of a first carrier substrate taken along section 5-5 in FIGS. 3 and 10.

FIG. 5A is a view of a first carrier substrate with leg cuffs and/or absorbent assemblies taken along section 5-5 in FIGS. 3 and 10.

FIG. 6 is a view of a second carrier substrate taken along section 6-6 in FIG. 3.

FIG. 6A is a view of a second carrier substrate with absorbent assemblies taken along section 6-6 in FIGS. 3 and 10.

FIG. 7 is a view of a continuous elastic substrate taken along section 7-7 in FIGS. 3 and 10.

FIG. 8 is a view of a discrete elastic part laid out flat with a zone of adhesive thereon taken along section 8-8 in FIG. 3.

FIG. 8A is a view of a discrete elastic part laid out flat with an alternate zone of adhesive thereon taken along section 8-8 in FIG. 3.

FIG. 9 is a view of a stretched discrete elastic part laid out flat bonded onto of the first carrier substrate along section 9-9 in FIGS. 3 and 10.

FIG. 9A is a view of a stretched discrete elastic part of FIG. 8A laid out flat bonded onto of the first carrier substrate along section 9-9 in FIGS. 3 and 10.

FIG. 10 is a schematic side view of an apparatus for bonding elastic parts to advancing first and second carrier webs.

FIG. 11 is a view of a cutting device, transfer device, and bonding device taken along section 11-11 in FIG. 10.

### DETAILED DESCRIPTION

The following term explanations may be useful in understanding the present disclosure:

“Absorbent article” is used herein to refer to consumer products whose primary function is to absorb and retain soils and wastes. Absorbent articles can comprise sanitary napkins, tampons, panty liners, interlabial devices, wound dressings, wipes, disposable diapers including taped diapers and diaper pants, inserts for diapers with a reusable outer cover, adult incontinent diapers, adult incontinent pads, and adult incontinent pants. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

“Relaxed state” defines a length of material when not stretched by an applied force.

In the context of the present description, an elongation of 0% refers to a material in relaxed state having a relaxed length of L, and elongation of 150% represents 2.5× the relaxed length, L, of the material. For example, an elastic film having a relaxed length of 100 millimeters would have a length of 250 millimeters at 150% elongation. And an elastic film having a relaxed length of 100 millimeters would have a length of 180 millimeters at 80% elongation.

In the context of the present description, a contraction of 60% represents 0.6× contraction of an initial stretch length, L, of a material. For example, an elastic film having an initial stretch length of 250 millimeters would have a contracted length of 100 millimeters at 60% contraction. And an elastic film having an initial stretch length of 180 millimeters would have a length of 100 millimeters at 44% contraction.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

The term “substrate” is used herein to describe a material which is primarily two-dimensional (i.e., in an XY plane) and whose thickness (in a Z direction) is relatively small (i.e., 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. In some configurations, a nonwoven may comprise a polyolefin based nonwoven, including but not limited to nonwovens having polypropylene fibers and/or polyethylene fibers and/or bicomponent fibers comprising a polyolefin. Nonlimiting examples of suitable fibers include spunbond, spunlaid, meltblown, spunmelt, solvent-spun, electrospun, carded, film fibrillated, melt-film fibrillated, air-laid, dry-laid, wet-laid staple fibers, and other nonwoven web materials formed in part or in whole of polymer fibers as known in the art, and workable combinations thereof. Nonwovens do not have a woven or knitted filament pattern. It is to be appreciated that nonwovens having various basis weights can be used in accordance with the methods herein.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

The term “taped diaper” (also referred to as “open diaper”) refers to disposable absorbent articles having an initial front waist region and an initial back waist region that are not fastened, pre-fastened, or connected to each other as packaged, prior to being applied to the wearer. A taped diaper may be folded about the lateral centerline with the interior of one waist region in surface to surface contact with the interior of the opposing waist region without fastening or joining the waist regions together. Example taped diapers are disclosed in various suitable configurations U.S. Pat. Nos. 5,167,897, 5,360,420, 5,599,335, 5,643,588, 5,674,216, 5,702,551, 5,968,025, 6,107,537, 6,118,041, 6,153,209, 6,410,129, 6,426,444, 6,586,652, 6,627,787, 6,617,016, 6,825,393, and 6,861,571; and U.S. Patent Publication Nos. 2013/0072887 A1; 2013/0211356 A1; and 2013/0306226 A1, which are all incorporated by reference herein.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, rear waist fastened or seamed). Example diaper pants in various configurations are disclosed in U.S. Pat. Nos. 4,940,464; 5,092,861; 5,246,433; 5,569,234; 5,897,545; 5,957,908; 6,120,487; 6,120,489; 7,569,039 and U.S. Patent Publication Nos. 2003/0233082 A1; 2005/0107764 A1, 2012/0061016 A1, 2012/0061015 A1; 2013/0255861 A1; 2013/0255862 A1; 2013/0255863 A1; 2013/0255864 A1; and 2013/0255865 A1, which are all incorporated by reference herein.

Aspects of the present disclosure relate to absorbent articles with a waist panel and a pocket positioned between a topsheet and a backsheet. As discussed in more detail below, an absorbent article may comprise: a chassis and a waist panel connected with the chassis. The chassis may comprise a topsheet, a backsheet, and an absorbent core positioned between the topsheet and the backsheet. The waist panel may comprise an inboard lateral edge, an outboard lateral edge, a first surface, and an opposing second surface. The first surface of the waist panel is bonded with a garment facing surface of the topsheet. A first region of the second surface of the waist panel is bonded with a wearer facing surface of the backsheet, and a second region of the second surface of the waist panel is unattached to the backsheet to define a pocket between the second region of the second surface of the waist panel and the backsheet. The pocket comprises an opening defined by a slit in the topsheet. In a method for assembling absorbent articles, a first carrier substrate may be advanced in a machine direction. An elastic part comprising a first surface and an opposing second surface may be provided. The first surface of the elastic part is bonded with the first carrier substrate, and a slit is cut into the first carrier substrate. The elastic part is sandwiched between the first carrier substrate and a second carrier substrate. A first region of the second surface the elastic part is bonded with the second carrier substrate, and a second region of the second surface of the elastic part remains unattached to the second carrier substrate to define a pocket between the second region of the second surface of the elastic part and the second carrier substrate. In turn, the slit in the first carrier substrate defines an opening of the pocket.

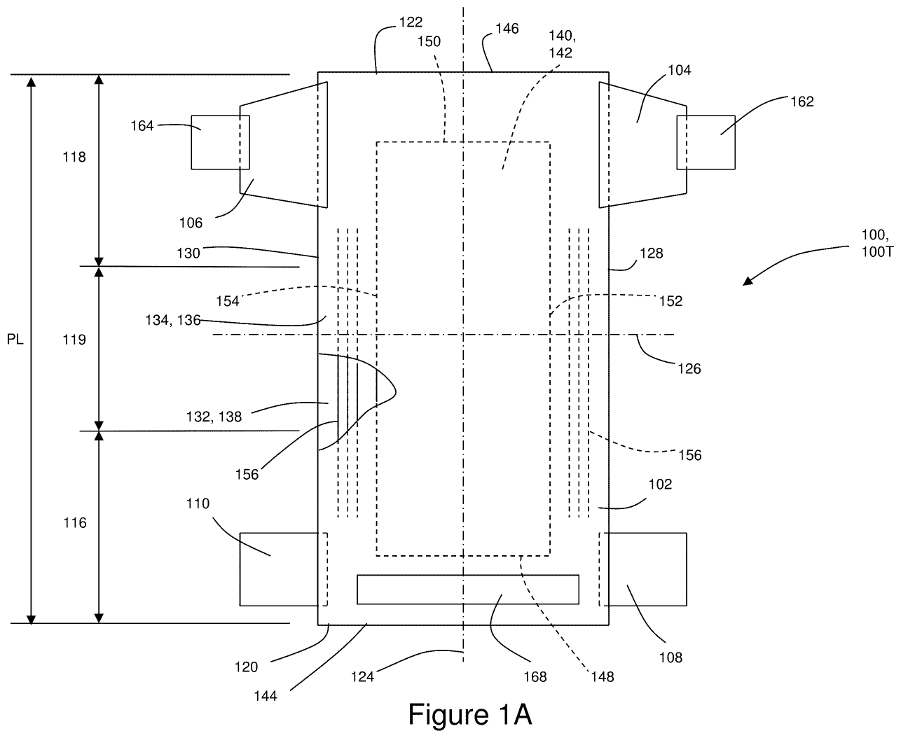

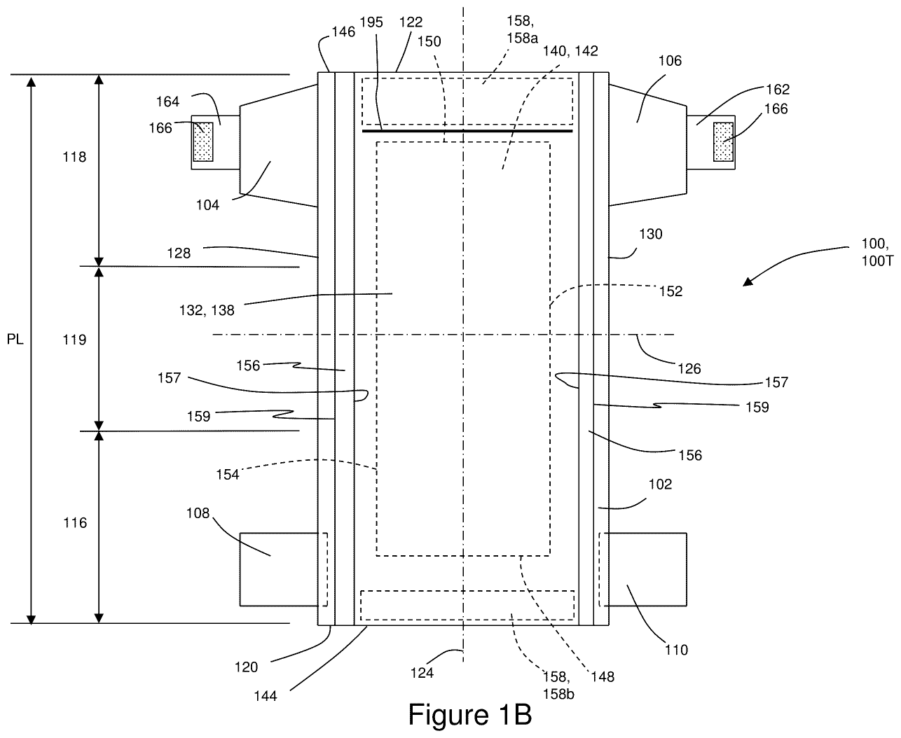

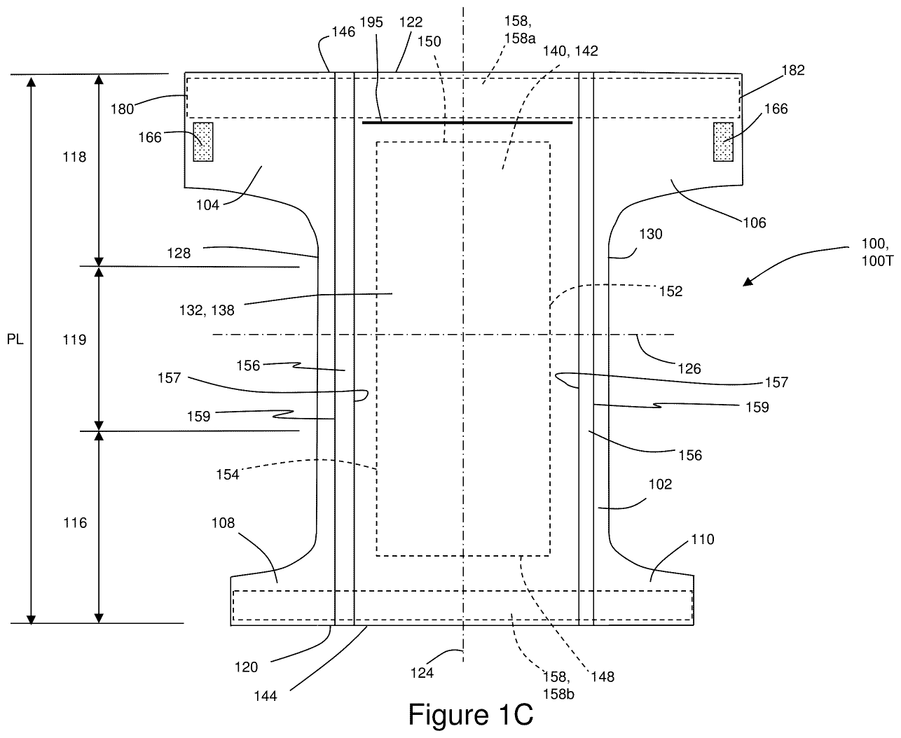

For the purposes of a specific illustration, FIGS. 1A and 1B show an example of an absorbent article 100 that may be assembled in accordance with the present disclosure. In particular, FIG. 1A shows one example of a plan view of an absorbent article 100 configured as a taped diaper 100T, with the portion of the diaper that faces away from a wearer oriented towards the viewer. And FIG. 1B shows a plan view of the diaper 100 with the portion of the diaper that faces toward a wearer oriented towards the viewer. The taped diaper 100T shown in FIGS. 1A and 1B includes an absorbent chassis 102, first and second rear side panels 104 and 106; and first and second front side panels 108 and 110. Although much of the following discussion is made with reference to an example taped diaper configuration, it is to be appreciated that the component configurations and methods herein may be adapted for application to diaper pant configurations.

As shown in FIGS. 1A and 1B, the absorbent article 100 and the chassis 102 each include a first waist region 116, a second waist region 118, and a crotch region 119 disposed intermediate the first and second waist regions. The first waist region 116 may be configured as a front waist region, and the second waist region 118 may be configured as a back waist region. In some configurations, the length of each of the front waist region, back waist region, and crotch region may be ⅓ of the length of the absorbent article 100. The absorbent article 100 may also include a laterally extending first waist edge 120 in the first waist region 116, wherein the first waist edge 120 may be configured as a front waist edge. In addition, the absorbent article 100 may include a laterally extending second waist edge 122 in the second waist region 118, wherein the second waist edge 122 may be configured as a back waist edge. To provide a frame of reference for the present discussion, the diaper 100T in FIGS. 1A and 1B is shown with a longitudinal axis 124 and a lateral axis 126. The longitudinal axis 124 may extend through a midpoint of the front waist edge 120 and through a midpoint of the back waist edge 122. And the lateral axis 126 may extend through a midpoint of a first longitudinal or right side edge 128 and through a midpoint of a second longitudinal or left side edge 130.

As shown in FIGS. 1A and 1B, the absorbent article 100 includes an inner, wearer facing surface 132, and an outer, garment facing surface 134. As such, it is also to be appreciated that the various components of the absorbent article described below may each include inner, wearer facing surfaces 132, and an outer, garment facing surfaces 134. The chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the absorbent article 100 may also include other features, such as leg gasketing elements, waist panels, and/or flaps, e.g., side panels and/or ears, to enhance the fits around the legs and waist of the wearer, to enhance the fit around the legs of the wearer.

As shown in FIGS. 1A and 1B, the periphery of the chassis 102 may be defined by the first longitudinal side edge 128, a second longitudinal side edge 130, a first laterally extending end edge 144 disposed in the first waist region 116, and a second laterally extending end edge 146 disposed in the second waist region 118. Both side edges 128 and 130 extend longitudinally between the first end edge 144 and the second end edge 146. As shown in FIG. 1A, the laterally extending end edges 144 and 146 may form a portion of the laterally extending front waist edge 120 in the front waist region 116 and a portion of the longitudinally opposing and laterally extending back waist edge 122 in the back waist region 118. The distance between the first lateral end edge 144 and the second lateral end edge 146 may define a pitch length, PL, of the chassis 102. When the absorbent article 100 is worn on the lower torso of a wearer, the front waist edge 120 and the back waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the front waist region 116 through the crotch region 119 to the back waist region 118.

It is to also be appreciated that a portion or the whole of the absorbent article 100 may also be made laterally extensible. The additional extensibility may help allow the absorbent article 100 to conform to the body of a wearer during movement by the wearer. The additional extensibility may also help, for example, the user of the absorbent article 100, including a chassis 102 having a particular size before extension, to extend the front waist region 116, the back waist region 118, or both waist regions of the absorbent article 100 and/or chassis 102 to provide additional body coverage for wearers of differing size, i.e., to tailor the absorbent article to an individual wearer. Such extension of the waist region or regions may give the absorbent article a generally hourglass shape, so long as the crotch region is extended to a relatively lesser degree than the waist region or regions, and may impart a tailored appearance to the article when it is worn.

As previously mentioned, the absorbent article 100 may include a backsheet 136. The backsheet 136 may also define the outer surface 134 of the chassis 102. The backsheet 136 may be impervious to fluids (e.g., menses, urine, and/or runny feces) and may be manufactured in part from a thin plastic film, although other flexible liquid impervious materials may also be used. The backsheet 136 may prevent the exudates absorbed and contained in the absorbent core from wetting articles which contact the absorbent article 100, such as bedsheets, pajamas and undergarments. The backsheet 136 may also comprise a woven or nonwoven material, polymeric films such as thermoplastic films of polyethylene or polypropylene, and/or a multi-layer or composite materials comprising a film and a nonwoven material (e.g., having an inner film layer and an outer nonwoven layer). The backsheet 136 may also comprise an elastomeric film. An example backsheet 136 may be a polyethylene film having a thickness of from about 0.012 mm (0.5 mils) to about 0.051 mm (2.0 mils). Exemplary polyethylene films are manufactured by Clopay Corporation of Cincinnati, Ohio, under the designation BR-120 and BR-121 and by Tredegar Film Products of Terre Haute, Ind., under the designation XP-39385. The backsheet 136 may also be embossed and/or matte-finished to provide a more clothlike appearance. Further, the backsheet 136 may permit vapors to escape from the absorbent core (i.e., the backsheet is breathable) while still preventing exudates from passing through the backsheet 136. The size of the backsheet 136 may be dictated by the size of the absorbent core 142 and/or particular configuration or size of the absorbent article 100.

Also described above, the absorbent article 100 may include a topsheet 138. The topsheet 138 may also define all or part of the inner surface 132 of the chassis 102. The topsheet 138 may be compliant, soft feeling, and non-irritating to the wearer's skin. It may be elastically stretchable in one or two directions. Further, the topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art.

Topsheets 138 may be selected from high loft nonwoven topsheets, apertured film topsheets and apertured nonwoven topsheets. Apertured film topsheets may be pervious to bodily exudates, yet substantially non-absorbent, and have a reduced tendency to allow fluids to pass back through and rewet the wearer's skin. Exemplary apertured films may include those described in U.S. Pat. Nos. 5,628,097; 5,916,661; 6,545,197; and 6,107,539, which are all incorporated by reference herein.

As mentioned above, the absorbent article 100 may also include an absorbent assembly 140 that is joined to the chassis 102. As shown in FIGS. 1A and 1B, the absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the absorbent article. Exemplary absorbent structures for use as the absorbent core of the present disclosure are described in U.S. Pat. Nos. 4,610,678; 4,673,402; 4,888,231; and 4,834,735, which are all incorporated by reference herein.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 A1 and 2004/0097895 A1, which are all incorporated by reference herein.

In some configurations, the absorbent assembly 140 may include an acquisition system disposed between the topsheet 138 and a wearer facing side of the absorbent core 142. The acquisition system may be in direct contact with the absorbent core 142 and may comprise a single layer or multiple layers, such as an upper acquisition layer (also referred to herein as a first acquisition layer) facing towards the wearer's skin and a lower acquisition layer (also referred to herein as a second acquisition layer) facing the garment of the wearer. In some configurations, the acquisition system may function to receive a surge of liquid, such as a gush of urine. As such, the acquisition system may serve as a temporary reservoir for liquid until the absorbent core 142 can absorb the liquid. Exemplary acquisition systems and associated manufacturing processes are described in U.S. Pat. Nos. 8,603,277 and 8,568,566; U.S. Patent Publication Nos. 2012/0316046 A1 and 2014/0163504 A1, all of which are hereby incorporated by reference herein.

In some configurations, the acquisition system may include chemically cross-linked cellulosic fibers. Such cross-linked cellulosic fibers may have various absorbency properties. Exemplary chemically cross-linked cellulosic fibers are disclosed in U.S. Pat. No. 5,137,537. Citric acid is an exemplary cross-linking agent. In some embodiments, polyacrylic acids may be used. In some configurations, the cross-linked cellulosic fibers may be crimped, twisted, or curled, or a combination thereof including crimped, twisted, and curled.

In some configurations, one or both of the upper acquisition layer and lower acquisition layer may include a nonwoven, which may be hydrophilic. Further, according to some configurations, one or both of the upper acquisition layer and lower acquisition layer may comprise chemically cross-linked cellulosic fibers, which may or may not form part of a nonwoven material. In some embodiments, the upper acquisition layer may comprise a nonwoven, without the cross-linked cellulosic fibers, and the lower acquisition layer may comprise the chemically cross-linked cellulosic fibers. Further, in some configurations, the lower acquisition layer may comprise the chemically cross-linked cellulosic fibers mixed with other fibers such as natural or synthetic polymeric fibers. According to some embodiments, such other natural or synthetic polymeric fibers may include high surface area fibers, thermoplastic binding fibers, polyethylene fibers, polypropylene fibers, PET fibers, rayon fibers, lyocell fibers, and mixtures thereof.

Exemplary absorbent assemblies 140, absorbent cores 142, and associated components that may be adapted for use with the present disclosure are described in U.S. Pat. Nos. 4,610,678; 4,673,402; 4,888,231; 4,834,735; 4,888,231; 5,260,345; 5,387,207; 5,397,316; 8,603,277; and 8,568,566; and U.S. Patent Publication Nos. 2012/0316046 A1 and 2014/0163504 A1, all of which are hereby incorporated by reference herein.

Taped diapers may be manufactured and provided to consumers in a configuration wherein the front waist region and the back waist region are not fastened, pre-fastened, or connected to each other as packaged, prior to being applied to the wearer. For example, the taped diaper 100T may be folded about a lateral centerline with the interior surface 132 of the first waist region 116 in surface to surface contact with the interior surface 132 of the second waist region 118 without fastening or joining the waist regions together. The rear side panels 104 and 106 and/or the front side panels 108 and 110 may also be folded laterally inward toward the inner surfaces 132 of the waist regions 116 and 118.

The absorbent article 100 may also include various configurations of fastening elements to enable fastening of the front waist region 116 and the back waist region 118 together to form a closed waist circumference and leg openings once the absorbent article is positioned on a wearer. For example, as shown in FIGS. 1A and 1B, the absorbent article 100 may include first and second fastening members 162, 164, also referred to as tabs, connected with the first and second rear side panels 104, 106, respectively. The absorbent article may also include first and second front side panels 108, 110, that may or may not include fastening members.

With continued reference to FIGS. 1A and 1B, each side panel 104, 106 and/or fastening member 162 and 164 may form a portion of or may be permanently bonded, adhered or otherwise joined directly or indirectly to the chassis 102 laterally inward from the side edge 128 and 130, in one of the front waist region 116 or the back waist region 118. Alternatively, the fastening members 162, 164 may form a portion of or may be permanently bonded, adhered or otherwise joined directly or indirectly to the first and second rear panels 104, 106 at or adjacent the distal edge of the panel and/or the first and second front side panels 108 and 110 at or adjacent the distal edge of the side panel. It is to be appreciated that the fastening members and/or side panels may be assembled in various ways, such as disclosed for example, in U.S. Pat. No. 7,371,302, which is incorporated by reference herein. The fastening members 162, 164 and/or side panels 104, 106, 108, 110 may also be permanently bonded or joined at or adjacent the side edges 128 and 130 of the chassis 102 in various ways, such as for example, by adhesive bonds, sonic bonds, pressure bonds, thermal bonds or combinations thereof, such as disclosed for example, U.S. Pat. No. 5,702,551, which is incorporated by reference herein. In some configurations, with the first and second rear side panels 104, 106 and/or the first and second front side panels 108, 110 may be integrally formed regions of the chassis 102, such as shown in FIG. 1C, wherein the topsheet 138 and backsheet 136 are formed with relatively narrow crotch regions 119 so as to define the shapes of the first and second rear side panels 104, 106.

Referring now to FIG. 1B, the first fastening member 162 and/or the second fastening member 164 may include various types of releasably engageable fasteners. The first and second fastening members 162 and/or 164 may also include various types of refastenable fastening structures. For example, the first and second fastening members 162 and 164 may include mechanical fasteners, 166, in the form of hook and loop fasteners, hook and hook fasteners, macrofasteners, buttons, snaps, tab and slot fasteners, tape fasteners, adhesive fasteners, cohesive fasteners, magnetic fasteners, hermaphroditic fasteners, and the like. Some examples of fastening systems and/or fastening members 162, 164 are discussed in U.S. Pat. Nos. 3,848,594; 4,662,875; 4,846,815; 4,894,060; 4,946,527; 5,151,092; 5,221,274; 5,242,436; 6,251,097; 6,669,618; 6,432,098; and U.S. Patent Publication Nos. 2007/0078427 A1; 2007/0093769 A1; and 2021/0145661 A1, which are all incorporated by reference herein.

As previously mentioned, the fastening members 162 and 164 may be constructed from various materials and may be constructed as a laminate structure. The fastening members 162 and 164 may also be adapted to releasably and/or refastenably engage or connect with another portion of the absorbent article 100. For example, as shown in FIG. 1A, the absorbent article 100 may include a connection zone 168, sometimes referred to as a landing zone, in the first waist region 116. As such, when the taped absorbent article 100 is placed on a wearer, the fastening members 162 and 164 may be pulled around the waist of the wearer and connected with the connection zone 168 in the first waist region 116 to form a closed waist circumference and a pair of laterally opposing leg openings. It is to be appreciated that the connection zone may be constructed from a separate substrate that is connected with the chassis 102 of the absorbent article. In some embodiments, the connection zone may be integrally formed as part of the backsheet 136 of the absorbent article 100 or may be formed as part of the first and second front panels 108, 110, such as described in U.S. Pat. Nos. 5,735,840 and 5,928,212, which are both incorporated by reference herein.

With continued reference to FIG. 1B, the absorbent article 100 may also include leg gasketing elements 156. It is to be appreciated that the leg gasketing elements 156 can be and are sometimes also referred to as leg cuffs, leg bands, side flaps, barrier cuffs, elastic cuffs or gasketing cuffs. The leg gasketing elements 156 may be elasticized and may be configured in various ways to help reduce the leakage of body exudates in the leg regions. Example leg gasketing elements 156 may include those described in U.S. Pat. Nos. 3,860,003; 4,909,803; 4,695,278; 4,795,454; 4,704,115; and U.S. Patent Publication No. 2009/0312730 A1, which are all incorporated by reference herein.

As shown in FIG. 1B, the absorbent article 100 may include longitudinally extending and laterally opposing leg gasketing elements 156 that are disposed on the interior surface 132 of the chassis 102 that faces inwardly toward the wearer and contacts the wearer. Each leg gasketing element 156 may have a first side edge 157 and a second side edge 159, wherein the first side edge 157 is positioned laterally inboard of the second side edge 159. The leg gasketing elements 156 may also overlap the absorbent assembly 140, wherein the first side edges 157 extend laterally inward of the respective side edges 152, 154 of the absorbent assembly 140. In some configurations, the leg gasketing elements 156 may not overlap the absorbent assembly 140. It is to be appreciated that the leg gasketing elements 156 may be formed in various ways, such as for example, by folding portions of the chassis 102 laterally inward, i.e., toward the longitudinal axis 124, to form both the respective leg gasketing elements and the side edges 128 and 130 of the chassis 102. In another example, the leg gasketing elements 156 may be formed by attaching an additional layer or layers to the chassis 102 at or adjacent to each of the respective side edges and of the chassis. Each of the leg gasketing elements 156 may be joined to the interior surface 132 of the chassis and/or the absorbent assembly 140 in leg gasketing element attachment zones in the front waist region 116 and in leg gasketing element attachment zones in the back waist region 118. The leg gasketing elements 156 may extend to the same longitudinal extent as the absorbent article 100 or alternatively the leg gasketing elements 156 may have a longitudinal extent that is less than the absorbent article 100. In some configurations, the leg gasketing elements may be configured to define inner cuffs, outer cuffs, or both inner and outer cuffs.

The absorbent article 100 may also include one or more waist panels 158, such as shown in FIG. 1B. The waist panel 158 may provide improved fit and containment and may define a portion or zone of the absorbent article 100 that may elastically expand and contract to dynamically fit a wearer's waist. In some configurations, the absorbent article 100 may include a single waist panel 158, which may be positioned in the second waist region. In some configurations, the absorbent article 100 may include more than one waist panels 158. For example, the absorbent article shown in FIG. 1B comprises a first waist panel 158a positioned in the second waist region 118 and a second waist panel 158b positioned in the first waist region 116. The waist panel 158 may be constructed in a number of different configurations including those described in U.S. Pat. Nos. 4,515,595 and 5,151,092; U.S. patent application Ser. No. 18/492,918; and U.S. Patent Publication Nos. 2020/0375807 A1; 2020/0375815 A1; 2021/0128366 A1; and 2021/0128369 A1, which are all incorporated by reference herein.

It is to be appreciated that the waist panels 158 herein may be configured in various ways and may include one or more elastic materials, such as for example, elastic film and/or strands. For example, the waist panel 158 may be configured as a single layer of elastic film. In some configurations, the waist panel 158 may be configured as a laminate of two more substrates. For example, the waist panel 158 may be configured as an elastic film and/or elastic strands bonded in between two or more nonwoven substrates and/or may be bonded with one or more nonwoven substrates. For example, the waist panel 158 may be configured as a bi-laminate with an elastic film bonded and/or elastic strands bonded with a single nonwoven substrate. In another example, the waist panel 158 may be configured as an elastic film and/or elastic strands bonded between two or more substrates, wherein the substrates may comprise nonwovens. It is also to be appreciated that nonwoven substrates of the waist panel 158 may be of the same or different material and/or basis weights and may be configured as an elastomeric nonwoven or a non-elastic nonwoven. In some configurations, one more nonwoven substrates of the waist panel 158 may be of the same or different material and/or basis weights as one more nonwoven substrates of the topsheet 138, backsheet 136, and/or leg gasketing elements 156.

It is to be appreciated that components of the waist panel 158 may be bonded together in various ways, such as for example, by adhesive bonds, ultrasonic bonds, pressure bonds, thermal bonds or combinations thereof. It is to be appreciated that components of the waist panel 158 may be bonded together with adhesive applied in various ways, such as for example, as a spray nozzle and/or a slot coating device. In some configurations, components of the waist panel 158 may be continuously bonded with adhesive or bonded discontinuously with a patterned adhesive. In some configurations, the adhesive may be applied in accordance with the apparatuses and/or methods disclosed in U.S. Pat. Nos. 8,186,296; 9,265,672; 9,248,054; and 9,295,590 and U.S. Patent Publication No. 2014/0148773 A1, which are all incorporated by reference herein. In some configurations, components of the waist panel 158 may be mechanically (pressure) bonded with the application of pressure (and optionally heat) in various ways, such as for example, the mechanical bonding devices and methods disclosed in in U.S. Pat. Nos. 4,854,984; 6,248,195; 8,778,127; 9,005,392; 9,962,297; and 10,052,237, which are all incorporated by reference herein. In some configurations, components of the waist panel 158 may be mechanically (pressure) bonded with the use of ultrasonic bonding methods configured in various ways, such as for example linear or rotary type configurations, and such as disclosed for example in U.S. Pat. Nos. 3,113,225; 3,562,041; 3,733,238; 5,110,403; 6,036,796; 6,508,641; and 6,645,330, which are all incorporated by referenced herein. It is to be appreciated that the waist panel 158 may be formed with various types of bond configurations, such as disclosed, for example, in U.S. Pat. Nos. 6,572,595; 6,830,800; 7,087,287; and 7,803,244; and U.S. Patent Publication Nos. 2018/0042778 A1; 2018/0042787 A1; 2018/0042779 A1; and 2018/0042780 A1, which are all incorporated by reference herein.

In some configurations, the waist panel 158 may be formed as a zero strain stretch laminate that may be connected with the chassis 102 in a stretched state. In some configurations, the zero strain stretch laminate may include at least a layer of nonwoven material and an elastomeric element. The elastomeric element may be attached to the layer of nonwoven material while in a relaxed or substantially relaxed state, and the resulting laminate is made stretchable (or more stretchable over a further range) by subjecting the laminate to an activation process, which elongates the nonwoven layer permanently and elongates the elastomeric element temporarily. In some configurations, the nonwoven layer may be a separate component, in which case the elastomeric element is attached to the nonwoven layer to form the laminate, which is then connected with the chassis 102. In some configurations, the nonwoven layer may be integral with at least a portion of the chassis 102, in which case the elastomeric element may be attached to the nonwoven layer and the nonwoven/elastomeric element laminate is subsequently activated. In some configurations, the waist panel may be an extrusion bonded laminate. If one or more layers of the waist panel 158 are provided separately, the waist panel 158 may be activated either before or after attachment to the chassis 102. Examples of zero strain activation processes are disclosed in U.S. Pat. Nos. 5,167,897 and 5,156,793, which are incorporated by reference herein.

It is to be appreciated that the waist panel 158 may be located in various positions relative to the wearer facing surfaces 132 and garment facing surfaces 134 of various absorbent article components. In some configurations, the waist panel 158 may be positioned in between the topsheet 138 and the backsheet 136. For example, as discussed in more detail below, the waist panel 158 may be positioned on the garment facing surface 134 of the topsheet 138 and the wearer facing surface 132 and the wearer facing surface 132 of the backsheet 136. As further described below, the waist panel 158 may be bonded with the topsheet 138 and the backsheet 134 so as to form a pocket 194 between the waist panel 158 and the backsheet 136. In addition, the pocket may comprise an opening defined by a slit 195 in the topsheet 138. In some configurations, the waist panel 158 may be positioned entirely longitudinally outboard of the absorbent assembly 140. And in some configurations, a portion of the waist panel 158 may overlap a portion of the wearer facing surface 132 of the absorbent assembly. As such, in some configurations, a portion of the pocket 194 may be formed between the waist panel 158 and the absorbent assembly 140. In addition, the slit 195 in the topsheet 138 may extend across the absorbent assembly 140 such that a portion of the absorbent assembly 140 may be positioned between the slit 195 in the topsheet 138 and the backsheet 136.

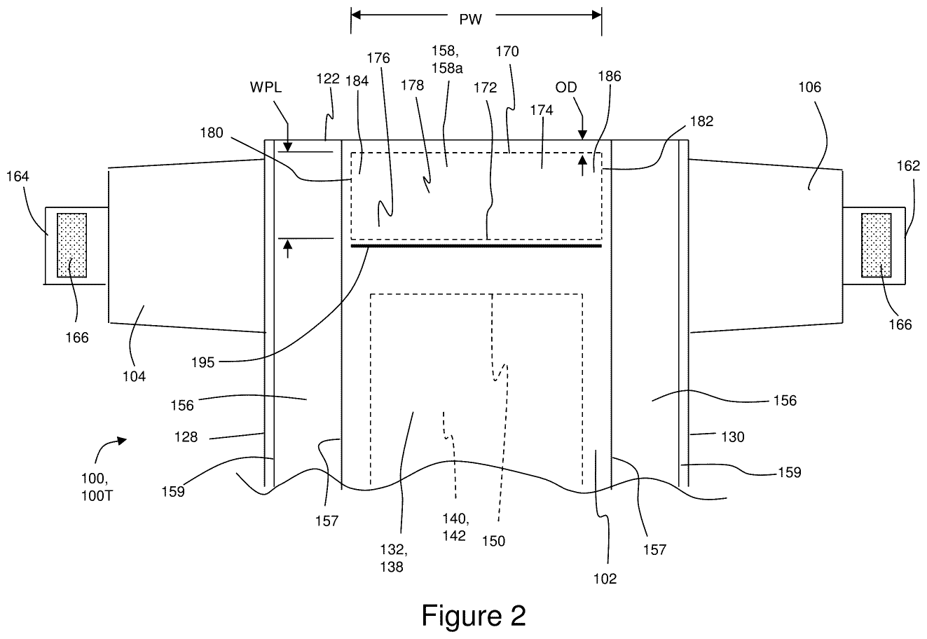

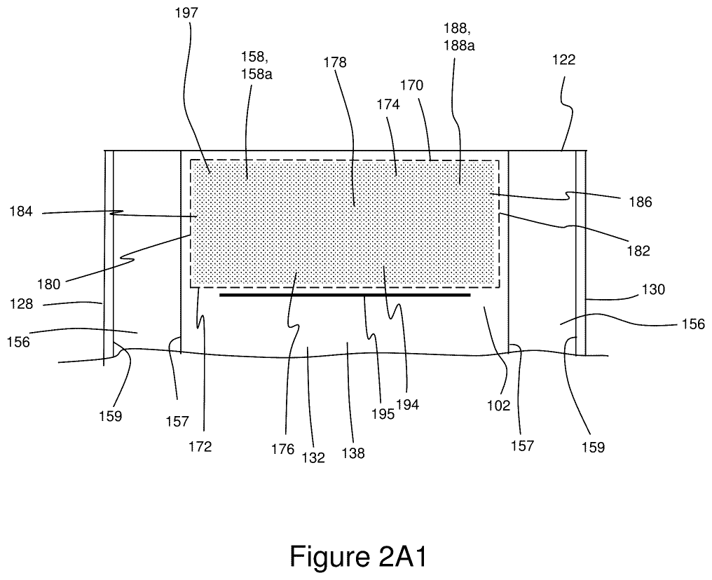

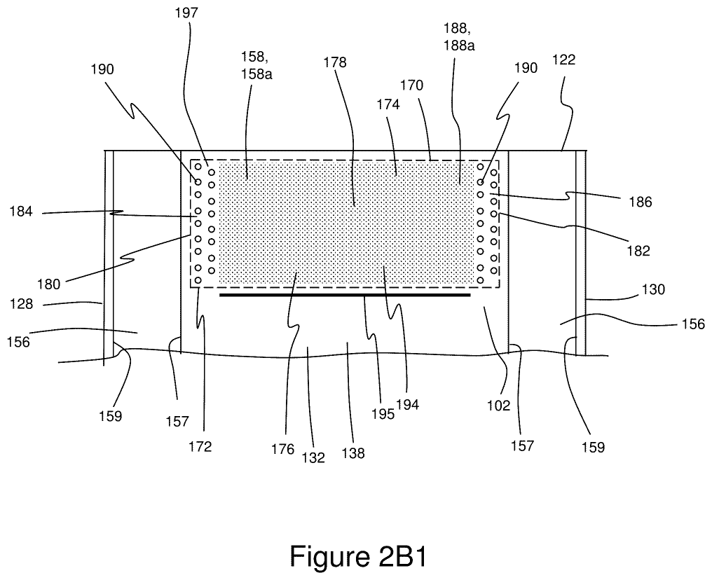

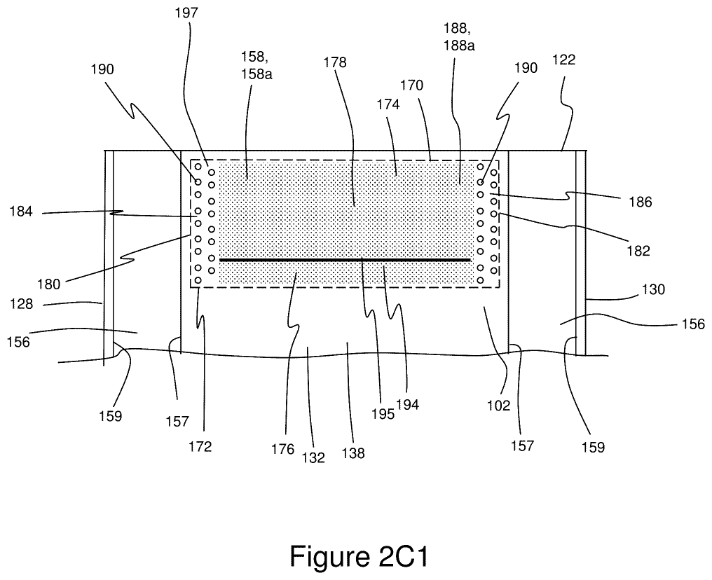

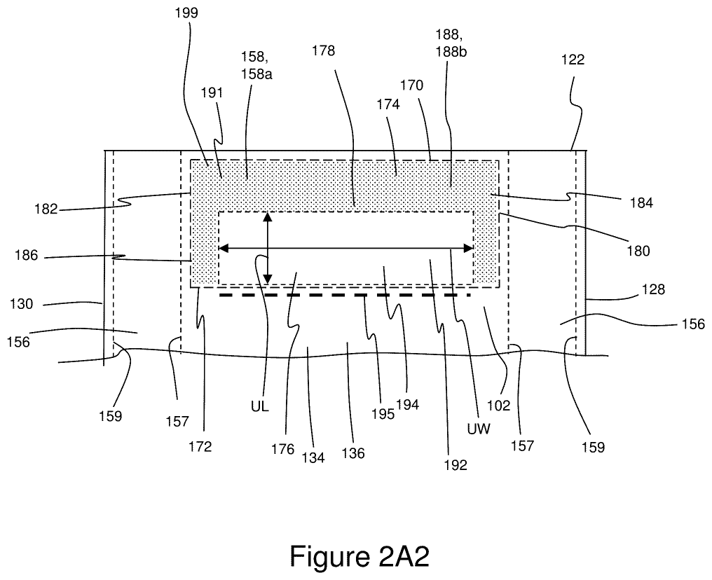

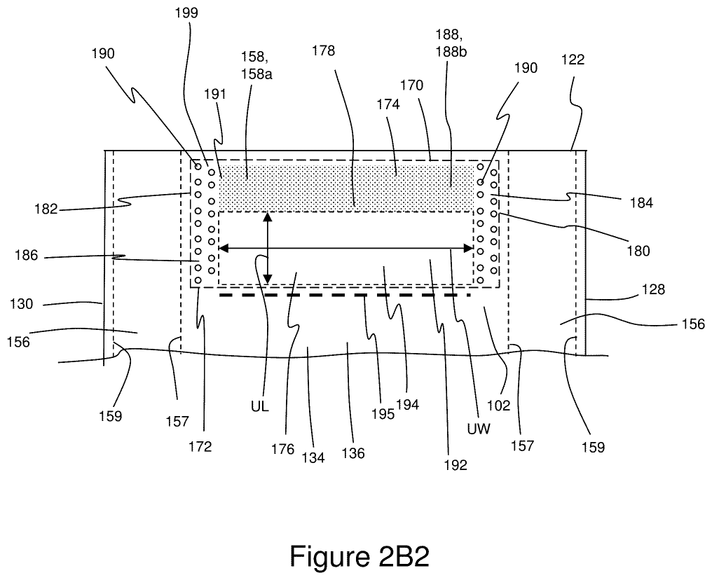

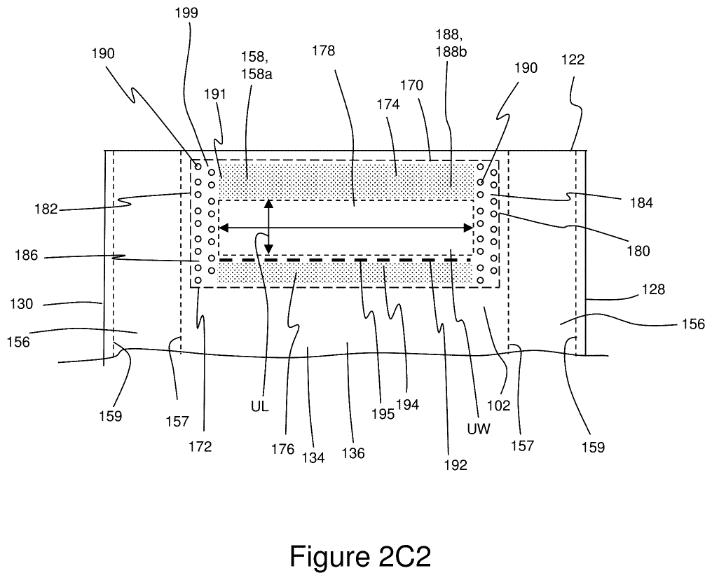

As shown in FIG. 2, the waist panels 158 herein may comprise a first lateral edge 170 and a second lateral edge 172, wherein the second lateral edge 172 is positioned longitudinally inward relative the first lateral edge 170. As such, the first lateral edge 170 may be configured as an outboard lateral edge, and the second lateral edge 172 may be configured as an inboard lateral edge. In addition, the waist panels 158 may comprise a first longitudinal end region 174 adjacent the first lateral edge 170 and a second longitudinal end region 176 adjacent the second lateral edge 172, wherein the first and second longitudinal end regions 174, 176 are separated by a central region 178. The first and second lateral edges 170, 172 may be connected with and separated by a first longitudinal edge 180 and a second longitudinal edge 182. As such, the waist panels 158 may also include a first lateral end region 184 adjacent the first longitudinal edge 180 and a second lateral end region 186 adjacent the second longitudinal edge 182, wherein the first and second lateral end regions 184, 186 are separated by the central region 178. In some configurations, the first lateral edge 170, second lateral edge 172, first longitudinal edge 180, and/or second longitudinal edge 182 may be defined by a fold line, wherein one or more layers of waist panel 158 may have been folded onto itself or another layer during assembly. In some configurations, the first lateral edge 170, second lateral edge 172, first longitudinal edge 180, and/or second longitudinal edge 182 may be defined by unfolded edge or a cut line, wherein one or more layers of waist panel 158 may have been cut or trimmed during assembly. Although FIG. 2 illustrates the first waist panel 158a, it is to be appreciated that the second waist panel 158b (if existent) may also comprise the above-referenced aspects.

As discussed above, the waist panels 158 herein may be elastic and may comprise at least one direction of stretch. In some configurations, the direction of stretch may be laterally oriented between the first longitudinal edge 180 and the second longitudinal edge 182. It is also to be appreciated that the first waist panel 158a may comprise stretch characteristics that are the same or different from stretch characteristics of the second waist panel 158b. Such stretch characteristics may comprise a percent contraction or a percent elongation. In some configurations, the stretch characteristics of the first waist panel 158a may be the same or may vary between the first lateral edge 170 and the second lateral edge 172 and/or the between the first longitudinal edge 180 and the second longitudinal edge 182. And in some configurations, the stretch characteristics of the second waist panel 158b may be the same or may vary between the first lateral edge 170 and the second lateral edge 172 and/or the between the first longitudinal edge 180 and the second longitudinal edge 182.

It is to be appreciated that the waist panels 158 herein may be configured with various shapes and/or sizes. For example, as shown in FIG. 2, the waist panel 158 may comprise a width PW extending between first and second longitudinal edges 180, 182. It is to be appreciated that the width PW of first and second waist panels 158a, 158b may be equal or different. The first waist panels 158 may comprise a length WPL extending between first and second lateral edges 170, 172. It is to be appreciated that the length WPL of first and second waist panels 158a, 158b may be equal or different.

With continued reference to FIG. 2, it is to be appreciated that the waist panels 158 may be located in various lateral and longitudinal positions relative to various absorbent article components. In some configurations, the waist panel 158 may be positioned such that the first and second longitudinal edges 180, 182 of the waist panel 158 are located laterally inboard of the leg gasketing elements 156. In some configurations, the waist panel 158 may be positioned such that the first and second longitudinal edges 180, 182 and the first and second longitudinal end regions 174, 176 of the waist panel 158 overlap the leg gasketing elements 156. As such, in some configurations, portions of the topsheet 138 may be positioned between opposing end portions of the waist panel 158 and the leg gasketing elements 156. In some configurations, the waist panel 158 may be positioned such that the first and second longitudinal edges 180, 182 of the waist panel 158 are located laterally outboard of the leg gasketing elements 156. For example, such as shown in FIG. 1C, the longitudinal edges 180, 182 of the waist panel 158 may be laterally outboard of the leg gasketing elements 156 and may be coterminous with or laterally inboard of the side edges 128, 130 of the chassis 102.

In some configurations, the first waist panel 158a may be positioned longitudinally inboard from the second waist edge 122 of the absorbent article 100 and/or toward or overlapping the second lateral edge 150 of the absorbent assembly 140; and the second waist panel 158b may be positioned longitudinally inboard from the first waist edge 120 of the absorbent article 100 and/or toward or overlapping the first lateral edge 148 of the absorbent assembly 140. In some configurations, the first lateral edge 170 of the first waist panel 158a may be positioned longitudinally inboard from the second waist edge 122 by an offset distance OD that is greater than zero. In some configurations, the first lateral edge 170 of the second waist panel 158b may be positioned longitudinally inboard from the first waist edge 120 by an offset distance that is greater than zero. In some configurations, the offset distance OD may be at least 5 mm. In some configurations, the first lateral edge 170 of the first waist panel 158a may be coterminous with the second waist edge 122 such that the offset distance OD is zero. In some configurations, the first lateral edge 170 of the second waist panel 158b may be coterminous with the first waist edge 120 such that the offset distance OD2 is zero.

It is to be appreciated that the waist panels 158 may be bonded with the chassis 102 in various ways, such as for example, by adhesive bonds, ultrasonic bonds, pressure bonds, thermal bonds or combinations thereof. It is also to be appreciated that the waist panel 158 may be bonded with the chassis 102 with adhesive applied in various ways, such as for example, as a spray nozzle and/or a slot coating device. In some configurations, the waist panels 158 may be continuously bonded with the chassis 102 with adhesive or bonded discontinuously with a patterned adhesive. In some configurations, the adhesive may be applied in accordance with the apparatuses and/or methods disclosed in U.S. Pat. Nos. 8,186,296; 9,265,672; 9,248,054; and 9,295,590 and U.S. Patent Publication No. 2014/0148773 A1, which are all incorporated by reference herein. In some configurations, the waist panels 158 may be mechanically (pressure) bonded with the chassis 102 with the application of pressure (and optionally heat) in various ways, such as for example, the mechanical bonding devices and methods disclosed in in U.S. Pat. Nos. 4,854,984; 6,248,195; 8,778,127; 9,005,392; 9,962,297; and 10,052,237, which are all incorporated by reference herein. In some configurations, the waist panels 158 may be mechanically (pressure) bonded with the chassis 102 with the use of ultrasonic bonding methods configured in various ways, such as for example linear or rotary type configurations, and such as disclosed for example in U.S. Pat. Nos. 3,113,225; 3,562,041; 3,733,238; 5,110,403; 6,036,796; 6,508,641; and 6,645,330, which are all incorporated by reference herein.

As mentioned above, the waist panels 158 herein may be bonded between the topsheet 138 and the backsheet 134 with adhesive bonds 188. For example, as shown in FIGS. 2A1 and 2A2, the waist panel 158 may include a first surface 197 and an opposing second surface 199. As shown in FIG. 2A1, the first surface 197 of the waist panel 158 may be bonded with the topsheet 138 with first adhesive bonds 188a, which is generically illustrated by a shaded region. As shown in FIG. 2A2, the second surface 199 of the waist panel 158 may be bonded with the backsheet 136 with second adhesive bonds 188b, which is generically illustrated by a shaded region.

In some configurations, the waist panels 158 herein may be bonded between the topsheet 138 and the backsheet 134 with combinations of adhesive bonds 188 and pressure bonds 190. For example, as shown in FIGS. 2B1 and 2B2, the waist panel 158 may include a first surface 197 and an opposing second surface 199. As shown in FIG. 2B1, the first surface 197 of the waist panel 158 may be bonded with the topsheet 138 with first adhesive bonds 188a, which is generically illustrated by a shaded region. As shown in FIG. 2B2, the second surface 199 of the waist panel 158 may be bonded with the backsheet 136 with second adhesive bonds 188b, which is generically illustrated by a shaded region. As shown in FIGS. 2B1 and 2B2, the first and second lateral end regions 184, 186 of the waist panel 158 may be bonded with the topsheet 138 and the backsheet 136 with pressure bonds 190. In some configurations, the pressure bonds 190 may be a discontinuous pattern of discrete bond sites. It is to be appreciated that the discrete bond sites may define various sizes and shapes and may be separated from each other by various distances. For example, in some configurations, the discrete bond sites may be separated from each other by at least 0.2 mm. It is also to be appreciated that the discrete bond sites may cover various different sized areas of the waist panel. In some configurations, the first and second lateral end regions 184, 186 extending along the first and second longitudinal edges 180, 182 may be bonded with the chassis 102 with a continuous bond that defines a sealed edge.

As discussed above, the waist panel 158 may be bonded with the topsheet 138 and the backsheet 134 so as to form a pocket 194 between the waist panel 158 and the backsheet 136. As shown in FIGS. 2A2 and 2B2, one or more regions of second surface 199 of the waist panel 158 (referred to herein as bonded regions 191) may be bonded with the backsheet 136, and one or more regions of the second surface 199 of the waist panel 158 (referred to as unbonded regions 192) may not be bonded (unattached) with the backsheet 136, thereby forming a pocket 194 between the waist panel 158 and the backsheet 136.

As shown in FIGS. 2A2 and 2B2, the waist panel 158 may comprise bonded regions 191 wherein the first longitudinal end region 174, the first lateral end region 184, and the second lateral end region 186 of the first waist panel 158a are bonded with backsheet 136; and the waist panel 158 may comprise at least one unbonded region 192 (generically illustrated by a rectangle with a dashed border) wherein a portion of the second longitudinal end region 176 and at least a portion the second lateral edge 172 may be unattached to the backsheet 136 and/or leg gasketing elements 156. It is to be appreciated that the waist panels 158 herein may be configured with one or more unbonded regions with various shapes and/or sizes. For example, as shown in FIGS. 2A2 and 2B2, the unbonded region 192 may comprise a laterally extending width UW and a longitudinally extending length UL.

As previously discussed and as shown in FIGS. 2-2B2, the pocket 194 may comprise the slit 195 in the topsheet 138, which may define an opening of the pocket 194. It is to be appreciated that the slit 195 may be located in various positions relative to the waist panel 158 and other absorbent article components. For example, the slit 195 may be positioned adjacent to the second lateral edge 172 of the waist panel 158. In some configurations, the slit 195 may be positioned longitudinally inboard of the second lateral edge 172 of the waist panel 158. In some configurations, the slit 195 may be positioned to be longitudinally aligned and/or coterminous with the second lateral edge 172 of the waist panel 158. In some configurations, portions of the slit 195 may extend across the second lateral edge 172 of the waist panel 158. In some configurations, the slit 195 may be positioned longitudinally outboard of the second lateral edge 150 of the absorbent assembly 140. It is also to be appreciated that the slit 195 may be curved and/or straight and may extend for various lengths. For example, the slit 195 may extend laterally for a length that is equal to, shorter than, or longer than the second lateral edge 172 of the panel 158. For example, as shown in FIG. 1C, the slit 195 may be positioned laterally inboard of the leg gasketing elements 156 while the waist panel 158 may extend laterally outboard of the leg gasketing elements 156. In some configurations, the slit 195 may extend laterally for a length that is equal to, shorter than, or longer than the laterally extending width UW of the unbonded region 192. In some configurations, the slit 195 may be configured as a perforation. Forming the slit 195 as a perforation may provide a user the options to open the pocket and form a waist cuff (by tearing the perforation) or alternatively leave the perforation closed and use the product as a waistband (pocket remains unopened).

In some configurations, the slit 195 may extend through the waist panel 158. For example, the slit 195 may be positioned longitudinally outboard of the second lateral edge 172 of the waist panel 158, such as shown for example in FIGS. 2C1 and 2C2. As such, portions of the waist panel extending longitudinally between the slit 195 and the second lateral edge 172 may be bonded with the topsheet 138 and/or backsheet 134. Region between slit and second lateral edge 172 remains bonded with backsheet 136. For example, as shown in FIG. 2C1, the portion of the first surface 197 of the waist panel 158 between the slit 195 and the second lateral edge 172 may be bonded with the topsheet 138 with adhesive and/or pressure bonds. And as shown in FIG. 2C2, the portion of the second surface 199 of the waist panel 158 between the slit 195 and the second lateral edge 172 may be bonded with the backsheet 138 with adhesive and/or pressure bonds. Although the bonding configurations shown in FIGS. 2C1 and 2C2 comprise pressure bonds and adhesive bonds similar to that shown in FIGS. 2B1 and 2B2, it is to be appreciated that the bonding configuration may be configured to be similar to that that shown in FIGS. 2A1 and 2A2.

In some configurations, the slit 195 may be positioned longitudinally inboard of the second lateral edge 150 of the absorbent assembly 140 such the slit 195 may extend across the absorbent assembly 140. As such, in some configurations, a portion of the absorbent assembly 140 may be positioned between the slit 195 and the backsheet 136. In some configurations, the slit 195 may be positioned in a location adjacent the second lateral edge 150 of the absorbent assembly 140. In such configurations, it is to be appreciated that measures may be taken to help mitigate risks of absorbent material from the absorbent assembly 140 from unintentionally escaping the absorbent assembly 140 through the second lateral edge 150 and/or the slit 195. For example, an additional a cover patch may be installed to help cover and/or seal the second lateral edge 150 of the absorbent assembly 140.

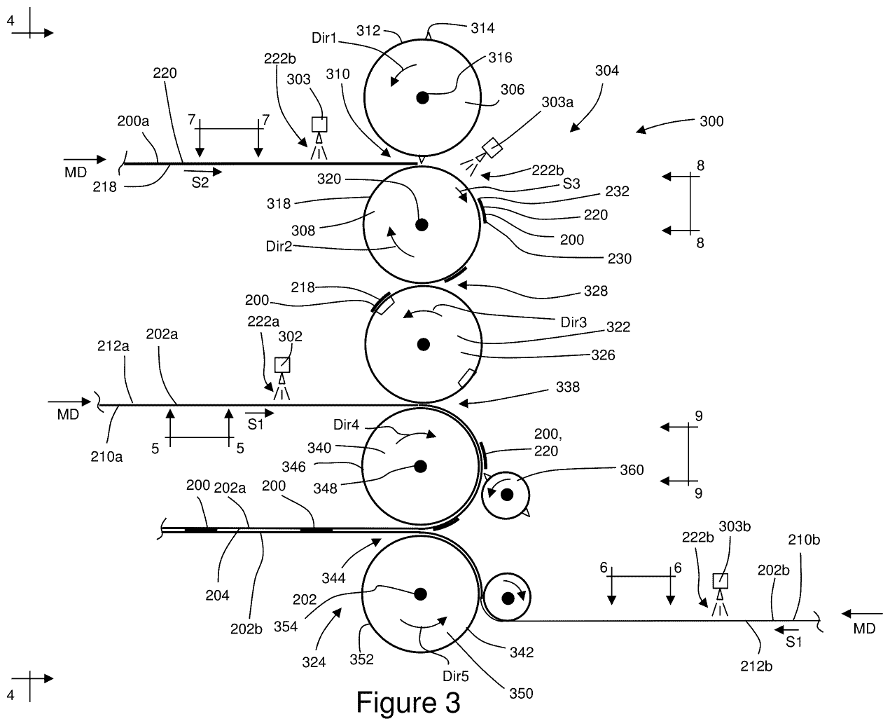

It is to be appreciated that absorbent articles 100 may be assembled with various components, including waist panels 158, described herein in various ways. Thus, in the context of the previous discussion, various apparatuses and methods may be adapted to assemble absorbent articles 100 with waist panels 158 bonded between topsheets 138 and backsheets 136 and to form a pocket 194 between the waist panel 194 and the backsheet 136. For example, FIG. 3 shows a schematic representation of a converting process including an apparatus or system 300 that bonds discrete elastic parts 200 under tension between an advancing first carrier substrate 202a and an advancing second carrier substrate 202b to form a laminate 204 during the assembly of an absorbent article 100. Various aspects of the apparatuses 300 and associated assemblies shown in FIG. 3 are disclosed in U.S. patent application Ser. No. 18/492,918; and U.S. Patent Publication Nos. 2020/0375807 A1; 2020/0375815 A1; 2021/0128366 A1; and 2021/0128369 A1, which are all incorporated by reference herein.

In the context of components of absorbent articles 100 discussed above and assembly processes thereof, the elastic parts 200 may be configured as waist panels 158. Although the following description is made in the context of discrete elastic parts 200 that are configured to correspond with the first waist panels 158a discussed above, it is to be appreciated that various alternatives are contemplated. For example, in some configurations, each discrete elastic part 200 may be configured as a first waist panel 158a, a second waist panel 158b, or may be a part that is subsequently cut along with the first and second carrier substrates 202a, 202b to be formed into a first waist panel 158a and a second waist panel 158b. The first carrier substrate 202a may be configured as a continuous topsheet 138, and the second carrier substrate 202b may be configured as a continuous backsheet 136. In turn, the laminate 204 may be configured as a continuous length of absorbent articles 100. As discussed above, the waist panels 158 and chassis 102 components may include various material combinations and constructions, and as such, it is to be appreciated that such combinations are also applicable to the elastic parts 200, and the first and second carrier substrates 202a, 202b.

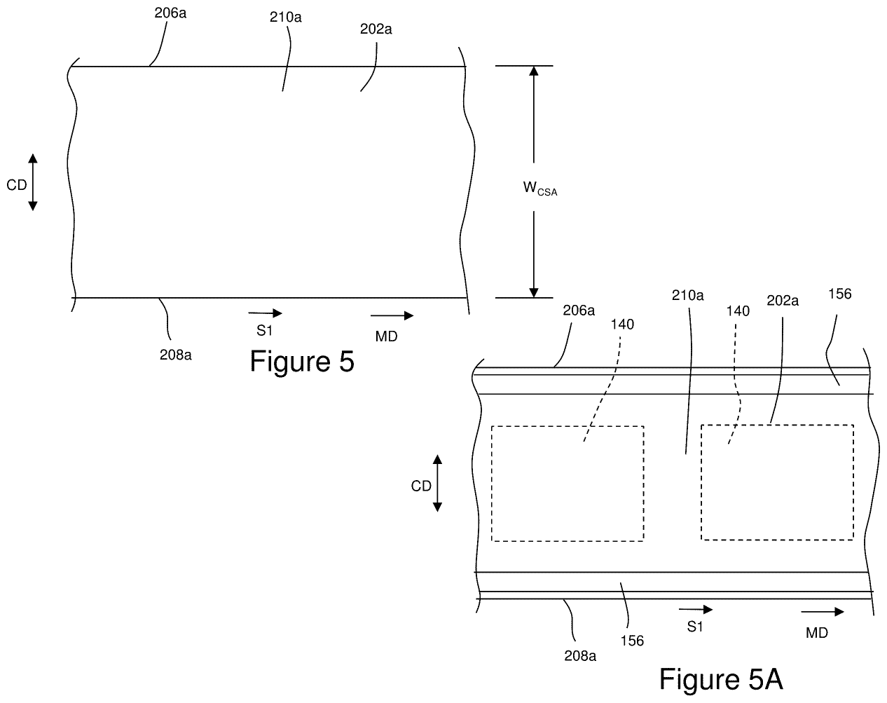

As shown in FIGS. 3 and 5, the first carrier substrate 202a may advance in a machine direction MD at a first speed S1. The first carrier substrate 202a comprises a first longitudinal edge 206a and a second longitudinal edge 208a separated from the first longitudinal edge 206a in a cross direction CD to define a width W_{CSA}. The carrier substrate 202 also includes a first surface 210a and an opposing second surface 212a. As discussed in more detail below, discrete elastic parts 200 are bonded with the second surface 212a of the first carrier substrate 202a, and a slit 195 is cut into the first carrier substrate 202a before being combined with the second carrier substrate 202b with the elastic parts 200 sandwiched between the first and second carrier substrates 202a, 202b. In some configurations, the first surface 210a of the first carrier substrate 202a may correspond with the wearer facing surface 132 of the topsheet 138, and the second surface 212a of the first carrier substrate 202a may correspond with the garment facing surface 134 of the topsheet 138.

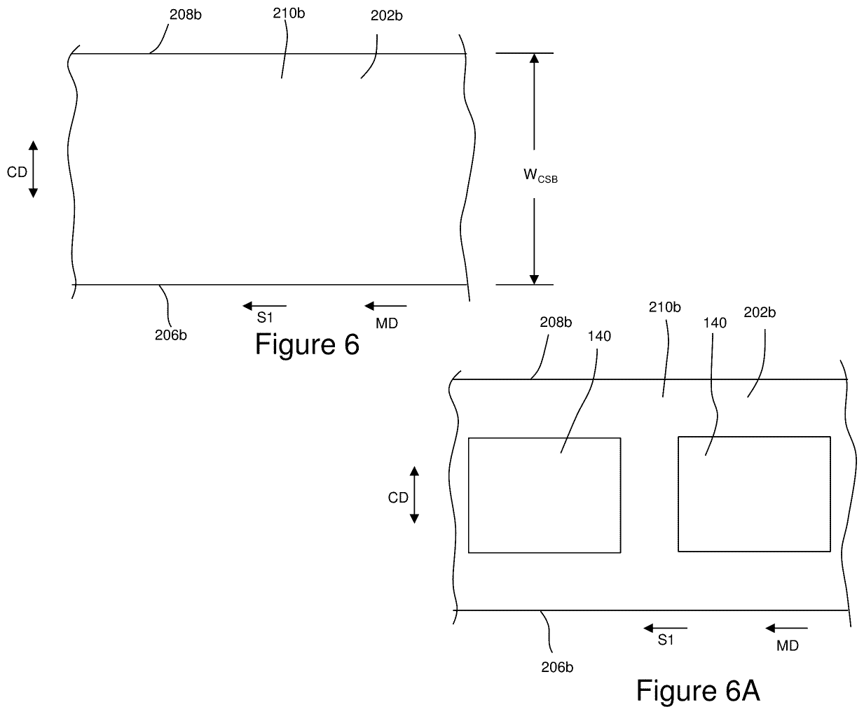

As shown in FIGS. 3 and 6, the second carrier substrate 202b may advance in a machine direction MD at the first speed S1. The second carrier substrate 202b comprises a first longitudinal edge 206b and a second longitudinal edge 208b separated from the first longitudinal edge 206b in a cross direction CD to define a width W_{CSB}. The second carrier substrate 202b also includes a first surface 210b and an opposing second surface 212b. As discussed in more detail below, the second carrier substrate 202b is combined with the first carrier substrate 202a with the elastic parts 200 sandwiched between the first and second carrier substrates 202a, 202b. In some configurations, the first surface 210b of the second carrier substrate 202b may correspond with the wearer facing surface 132 of the backsheet 136, and the second surface 212b of the second carrier substrate 202b may correspond with the garment facing surface 134 of the backsheet 136.

As shown in FIG. 5A, the first carrier substrate 202a may also include leg gasketing elements 156 positioned on the first surface 210a adjacent the first longitudinal edge 206a and the second longitudinal edge 208a. In some configurations, the discrete elastic parts 200 may be bonded with the first carrier substrate 202a and leg gasketing elements 156 may subsequently be bonded with the first carrier substrate 202a. With continued reference to FIG. 5A, the first carrier substrate 202a may also include absorbent assemblies 140 positioned on the second surface 212a. In some configurations, the discrete elastic parts 200 may be bonded with the first carrier substrate 202a and absorbent assemblies 140 may subsequently be bonded with the first carrier substrate 202a. As shown in FIG. 6A, the second carrier substrate 202b may also include absorbent assemblies 140 positioned on the second surface 210b.

Referring now to FIGS. 3 and 7, a continuous elastic substrate 200a is advanced at a second speed S2 in a machine direction MD, wherein the second speed S2 is less than the first speed S1. The continuous elastic substrate 200a comprises a first longitudinal edge 214 and a second longitudinal edge 216 separated from the first longitudinal edge 214 in the cross direction CD to define a width W_{ES}. The continuous elastic substrate 200a also includes a first surface 218 and an opposing second surface 220. The continuous elastic substrate 200a is stretchable in at least one direction and is oriented such that the continuous elastic substrate 200a is stretchable in the cross direction CD. As such, the width W_{ES} of the continuous elastic substrate may be an unstretched width. In some configurations, the width W_{ES} of the continuous elastic substrate 200a may be a partially stretched width.

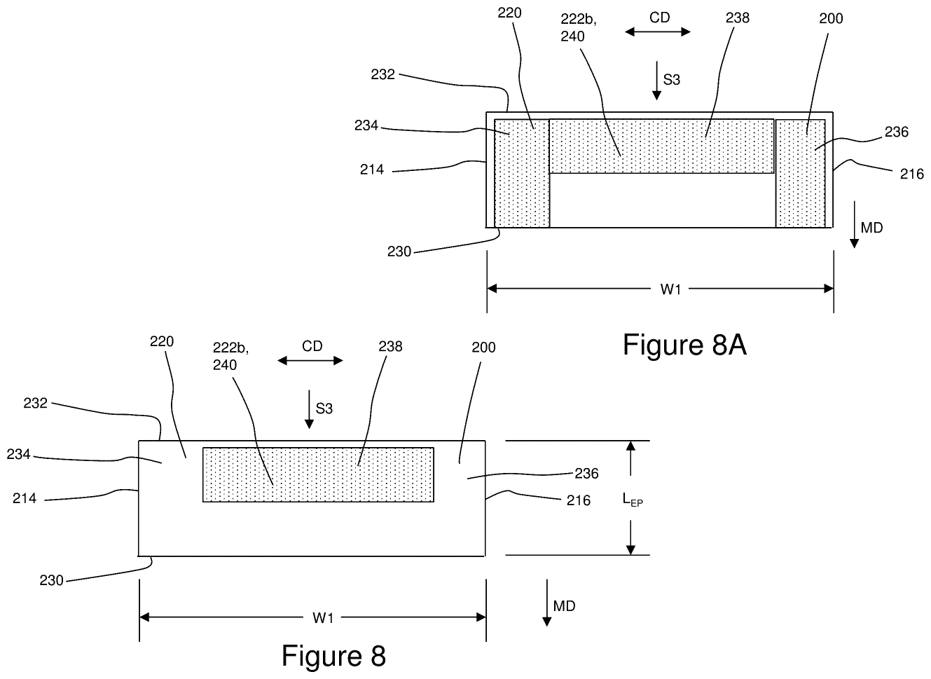

As shown in FIGS. 3 and 8, the continuous elastic substrate 200a may advance in the machine direction MD to a cutting device 304 that cuts and separates discrete elastic parts 200 from the continuous elastic substrate 200a. As such, the discrete elastic parts 200 each include a leading edge 230 and a trailing edge 232 and defines a length L_{EP} in the machine direction MD extending from the leading edge 230 to the trailing edge 232. The elastic part 200 also includes first and second longitudinal edges 214, 216 that correspond with the longitudinal edges 214, 216 of the continuous elastic substrate 200a extending between the leading and trailing edges 230, 232. In addition, the elastic part 200 includes first and second surfaces 218, 220 that correspond with the first and second surfaces 218, 220 of the continuous elastic substrate 200a. As shown in FIG. 7, the discrete elastic part 200 also includes a first end region 234 adjacent the first longitudinal edge 214 and a second end region 236 adjacent the second longitudinal edge 216, wherein the second end region 236 is separated from the first end region 234 in the cross direction CD by a central region 238. It is to be appreciated that the continuous elastic substrate 200a and discrete elastic parts 200 herein may be configured in various ways and may include one or more elastic materials, such as for example, elastic film and/or strands.

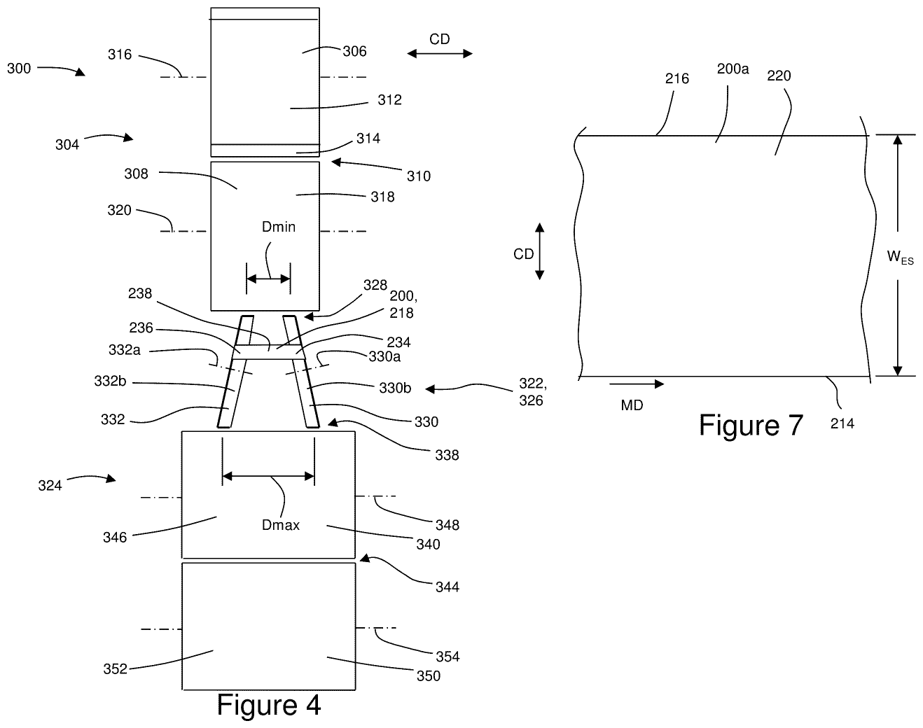

As shown in FIGS. 3 and 4, the cutting device 304 may include a knife roll 306 positioned adjacent an anvil roll 308 to define a nip 310 therebetween. The knife roll 306 may include an outer circumferential surface 312 and one or more blades 314 adapted to rotate about an axis 316 in a first direction Dir1. The anvil roll 308 may include an outer circumferential surface 318 adapted to rotate about an axis 320 in a second direction Dir2 opposite the first direction Dir1 such that the outer circumferential surface 318 advances at a third speed S3, wherein the third speed S3 is greater than the second speed S2. With continued reference to FIG. 3, as the continuous elastic substrate 200a advances through the nip 310 between the knife roll 306 and the anvil roll 310, the blade 314 operates to cut the discrete elastic part 200 from the continuous elastic substrate 200a. Because the outer circumferential surface 318 of the anvil roll 308 advances at the third speed S3, the cut discrete elastic part 200 may then accelerate from the second speed S2 to the third speed S3 on the outer circumferential surface 318 of the anvil roll 308. It is also to be appreciated that one or more components of the cutting device 304 may be configured to operate at constant and/or variable speeds. For example, the knife roll 306 and/or the anvil roll 308 may be connected with various types of motors, such as servo motors for example, that may rotate the knife roll 306 and/or the anvil roll 308 at constant and/or variable angular velocities.

In some configurations, the third speed S3 may be equal to the first speed S1 of the advancing first and second carrier substrates 202a, 202b. In some configurations, the third speed S3 may be less than or greater than the first speed S1 of the advancing first and second carrier substrates 202a, 202b, and as such, the discrete elastic part 200 may be accelerated or decelerated downstream of the anvil roll 308 from the third speed S3 to the first speed S1 before being combined with the first carrier substrate 202a. Because the first speed S1 of the first and second carrier substrates 202a, 202b is greater than the second speed S2, the discrete elastic parts 200 are accelerated from the second speed S2 to the first speed S1 before bonding with the first carrier substrate 202a. By accelerating discrete elastic parts 200 from the second speed S2 to the first speed S1, trailing edges 232 (or leading edges 230) of consecutively cut discrete elastic parts 200 may be separated from each other in the machine direction MD by a pitch distance, which may correspond with the pitch length PL described above with reference to FIGS. 1A and 1B. The anvil roll 308 may also be configured to apply vacuum pressure to the discrete elastic parts 200 to help hold the discrete elastic parts 200 on the outer circumferential surface 318 as the anvil roll 308 rotates.

It is to be appreciated that the cutting device 304 may be configured in various ways. For example, in some configurations, the blade 314 may be configured such that resulting cut lines and corresponding leading edges 230 and trailing edges 232 of the discrete elastic parts 200 may be straight and/or curved. The cutting device 304 may also be adapted to cut the discrete elastic parts 200 such that material along the cut line adjacent leading edges 230 and trailing edges 232 is fused and/or pressure bonded together. It is also to be appreciated that the positions of the knife roll 306 and anvil roll 308 may be opposite to that which is illustrated in FIG. 3, and as such, the discrete elastic parts 200 may remain on the outer circumferential surface 312 of the knife roll 306 as opposed to the anvil roll 308. It is also to be appreciated that the cutting device 304 may be configured to convey and/or cut the discrete elastic parts 200 in different ways.

With reference to FIG. 3, the apparatus 300 may include a rotatable transfer device 322 that transfers the discrete elastic parts 200 from the cutting device 304 to a bonding device 324, which in turn, combines the elastic parts 200 with the first carrier substrate 202a and the second carrier substrate 202b. The transfer device 322 may also be configured to stretch the discrete elastic parts 200 in the cross direction CD. In some configurations, the transfer device 322 may be configured as a spreader mechanism 326, such as shown in FIG. 4. With continued reference to FIGS. 3 and 4, the transfer device 322 may be positioned adjacent the anvil roll 308 to define a nip 328 therebetween. As discussed in more detail below, the discrete elastic parts 200 are received from the anvil roll 308 and the spreader mechanism 326 operates to stretch discrete elastic parts 200 in the cross direction CD. The stretched discrete elastic parts 200 are then advanced from the spreader mechanism 326 onto the first carrier substrate 202a advancing on the bonding device 324, which in turn, bonds the stretched discrete elastic parts 200 between the first carrier substrate 202a and the second carrier substrate 202b.

As shown in FIGS. 3 and 4, the spreader mechanism 326 may include a first disk 330 and a second disk 332, wherein the first disk 330 is displaced from the second disk 332 in the cross direction CD. The first disk 330 is adapted to rotate about an axis of rotation 330a and the second disk 332 is adapted to rotate about an axis of rotation 332a, wherein the first and second disks 330, 332 may rotate in a third direction Dir3 that is opposite the second direction Dir2. As shown in FIG. 4, the first disk 330 includes an outer rim 330b extending axially between an inner edge and an outer edge, and the second disk 332 includes an outer rim 332b extending axially between an inner edge and an outer edge.

As shown in FIG. 4, the first disk 330 and the second disk 332 are canted relative to each other such that the outer rims 330b, 332b are separated from each other by a distance D that increases from a minimum distance Dmin at a first location to a maximum distance Dmax at a second location. As discussed below, the discrete elastic parts 200 are transferred from the cutting device 304 onto the outer rims 330b, 332b during operation. Because the first and second disks 330, 332 are canted, rotation of the disks 330, 332 causes the rims 330b, 332b to pull on first end region 234 and the second end region 236 of discrete elastic parts 200 and stretch the central regions 238 of the discrete elastic parts 200 in the cross direction CD before the discrete elastic parts 200 are transferred to first carrier substrate 202a on the bonding device 324. As shown in FIGS. 3, 8, and 9, the spreader mechanism 326 may operate to stretch the discrete elastic parts 200 in the cross direction CD from a first width W1 to a second width W2 that is greater than the first width W1.

As previously mentioned, the rotatable transfer device 322 may be configured to transfer the discrete elastic parts 200 from the cutting device 304 to the first carrier substrate 202a on a bonding device 324. As shown in FIGS. 3 and 4, the bonding device 324 may be positioned adjacent the first and second disks 330, 332 of the spreader device 326 to define a nip 338 therebetween. In some configurations, the first and second disks 330, 332 may be configured to apply positive air pressure, sometimes referred to as blow-off air, to the discrete elastic part 200 adjacent the nip 338 to help remove the discrete elastic parts 200 from the disks 330, 332 during transfer to the first carrier substrate 202a on the bonding device 324.

As discussed above, the cut discrete elastic parts 200 accelerate from the second speed S2 to the third speed S3 on the outer circumferential surface 318 of the anvil roll 308, and in some configurations, the third speed S3 may be less than or greater than the first speed S1 of the advancing carrier substrate 202. Thus, the transfer device 322 may be configured to rotate at a variable angular velocity to accelerate or decelerate the discrete elastic parts 200 to the first speed S1. For example, if the third speed S3 is less than the first speed S1, the transfer device 322 may be configured to receive the discrete elastic part 200 from the anvil roll 308 while the rims 330b, 332b of the first and second disks 330, 332 are moving through the nip 328 at the third speed S3. The angular velocity of the disks 330, 332 may then be changed to accelerate the discrete elastic part 200 to the first speed S1 before transferring the discrete elastic part 200 to the bonding device 324. In another example, if the third speed S3 is greater than the first speed S1, the angular velocity of the disks 330, 332 may be changed to decelerate the discrete elastic part 200 to the first speed S1 before transferring the discrete elastic part 200 to the bonding device 324. In situations where the third speed S3 is equal to the first speed S1, the disks 330, 332 may rotate at a constant angular velocity. It is to be appreciated that the spreader mechanism 326 may be configured in various ways to accommodate a need to rotate at variable angular velocities, such as, for example, disclosed in European Patent Publication No. EP 2260813 B1, which is incorporated by reference herein. The ability to rotate at the transfer device 326 at variable angular velocities may help reduce the need to replace components of the apparatus 300 when assembling absorbent articles 100 of smaller or larger sizes, which in turn, may require a reduction or increase in the pitch distances between consecutively cut discrete elastic parts 200.

Referring again to FIG. 3, the system 300 may include a first adhesive applicator device 302 that deposits a first adhesive 222a onto the second surface 212a of the first carrier substrate 202a. The first adhesive 222a may be applied to bond the first surface of the elastic part 200 with the second surface 212a of the first carrier substrate 202a. As such, the first adhesive 222a may form the first adhesive bonds 188a discussed above with reference to FIGS. 2A1 and 2A2. The system 300 may also include a second adhesive applicator device 303 that deposits a second adhesive 222b onto the second surface 220 of the continuous elastic substrate 200a. As discussed in more detail below, the second adhesive 222b may be applied to bond the second surface of the elastic part 200 with the first surface 210b of the second carrier substrate 202b. As such, the second adhesive 222b may form the second adhesive bonds 188b discussed above with reference to FIGS. 2B1 and 2B2. It is to be appreciated that the adhesive applicator devices 302, 303 may be configured in various way, such as for example, as a spray nozzle and/or a slot coating device. In some configurations, the adhesive applicator devices 302, 303 may be configured in accordance with the apparatuses and/or methods disclosed in U.S. Pat. Nos. 8,186,296; 9,265,672; 9,248,054; and 9,295,590 and U.S. Patent Publication No. 2014/0148773 A1, which are all incorporated by reference herein.

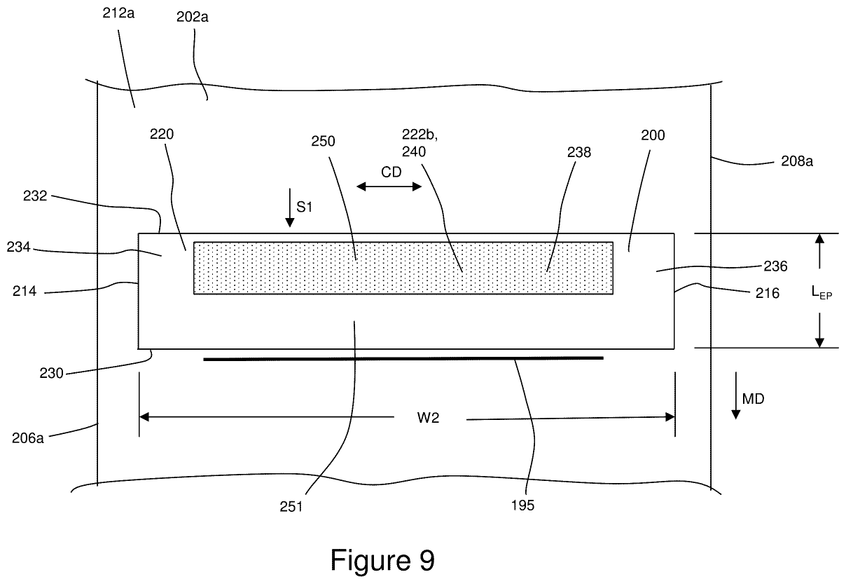

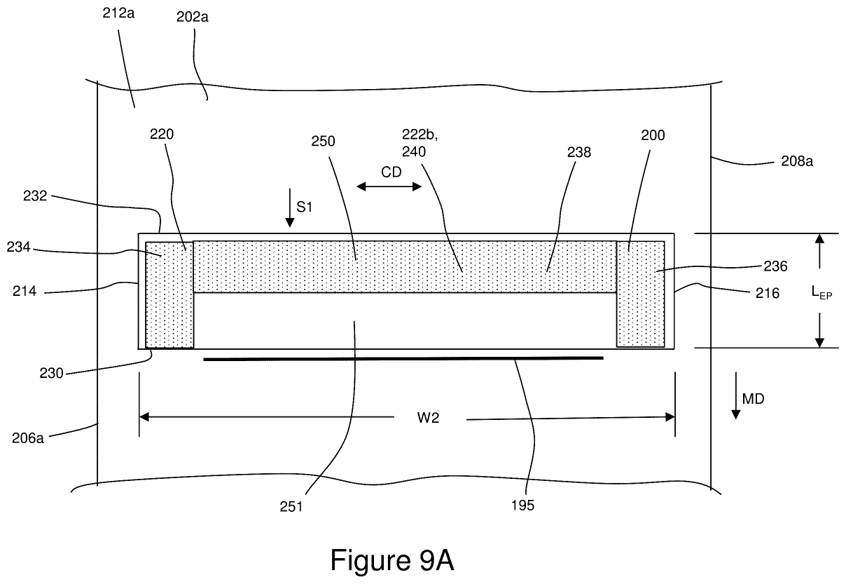

As shown in FIGS. 3 and 9, the first carrier substrate 202a may advance from the first adhesive applicator device 302 and through the nip 338 between the rotatable transfer device 322 and the bonding device 324. At the nip 338, the discrete elastic parts 200 are received from the transfer device 322 onto the second surface 212a of the first carrier substrate 202a with the central regions 238 stretched in the cross direction CD. In turn, the bonding device 324 bonds the discrete elastic parts 200 in the stretched state between the advancing first and second carrier substrates 202a, 202b.

As discussed above, the second adhesive applicator device 303 may apply the second adhesive 222b to the second surface 220 of the continuous elastic substrate 200a. As such, the discrete elastic part 200 may include a zone 240 of second adhesive 222b on the second surface 220. It is to be appreciated that the zone 240 of adhesive 222b may define various sizes and shapes relative to the elastic part 200, such as discussed above with reference to the second adhesive bonds 188b shown in FIGS. 2B1 and 2B2. For example, in some configurations, as shown in FIGS. 8 and 9, the zone 240 of second adhesive 222b may extend in the cross direction CD for less than the entire width W1 of the discrete elastic part 200. The zone 240 of second adhesive 222b may be positioned only on the central region 238 of the discrete elastic part 200 such that the first end region 234 and the second end region 236 of the second surface 220 of the discrete elastic part 200 may not include any adhesive 222b. In turn, the first end region 234 and the second end region 236 of the second surface 220 of the discrete elastic part 200 may be subsequently mechanically bonded with the second carrier substrate 202b with the pressure bonds 190 as discussed above for example. In some configurations, as shown in FIGS. 8A and 9A, the zone 240 of second adhesive 222b may extend in the cross direction CD for substantially the entire width W1 of the discrete elastic part 200. The zone 240 of second adhesive 222b may be positioned on the central region 238 of the discrete elastic part 200 as well as the first end region 234 and the second end region 236 of the second surface 220 of the discrete elastic part 200.

With continued reference to FIGS. 8, 8A, 9, and 9A, the zone 240 of second adhesive 222b may also be positioned in a first region 250 of the second surface 220 of the elastic part 200 adjacent the trailing edge 232 such that a second region 251 of the second surface 220 of the elastic part 200 adjacent the leading edge 230 may not include any second adhesive 222b. As discussed below, the first region 250 of the second surface 220 the elastic part 200 is bonded with the second carrier substrate 202b, and the second region 251 of the second surface 220 remains unattached to the second carrier substrate 202b to define the pocket 194 between the second region 251 of the second surface 220 of the elastic part 200 and the second carrier substrate 202b. It is also to be appreciated that the in some configurations, the zone 240 of second adhesive 222b may also be positioned adjacent the leading edge 230 of the elastic part 200 such that an area adjacent the trailing edge 232 of the discrete elastic part 200 may not include any second adhesive 222b.

As previously discussed with reference to FIG. 8, the elastic part 200 may include a zone 240 of second adhesive 222b that is positioned on the central region 238 of the discrete elastic part 200 and wherein portions of or all of the first end region 234 and the second end region 236 of the second surface 220 of the discrete elastic part 200 may not include any adhesive 222. As shown in FIGS. 3 and 4, once transferred to the transfer device 322, the elastic parts 200 may be oriented such that the first surface 218 may be facing radially outward, and the second surface 220 and the zone 240 of adhesive 222 may be facing radially inward. As such, the arrangement of disks 330, 322 of the spreader mechanism 326 provide the ability to rotatably convey the elastic parts 200 from the cutting device 304 to the bonding device 324 with a zone 240 of adhesive 222 that faces radially inward without having to contact the adhesive 222 with the disks 330, 332.

It is to be appreciated that the bonding device 324 may be configured in various ways. For example, as shown in FIGS. 3 and 4, the bonding device 324 may be configured with a pattern roll 340 and a pressing surface 342 adjacent the pattern roll 340 to define a nip 344 therebetween. The pattern roll 340 includes an outer circumferential surface 346 and rotates about an axis of rotation 348, wherein the pattern roll 340 may rotate in a fourth direction Dir4 that is opposite the third direction Dir3. In addition, pattern roll 340 may rotate such that the outer circumferential surface 346 advances at or about the first speed S1. During operation, the first carrier substrate 202a advances from the first adhesive applicator device 302 to the nip 338 and onto the outer circumferential surface 346 of the pattern roll 340. Discrete elastic parts 200 in a stretched state are transferred from the first and second disks 330, 332 to the second surface 212a of the first carrier substrate 202a at the nip 338. As such, the first adhesive 222a bonds the first surfaces 218 of the discrete elastic parts 200 with the second surface 212a of the first carrier substrate 202a.

With continued reference to FIG. 3, the pattern roll 340 rotates to advance first carrier substrate 202a and the stretched elastic parts 200 bonded thereto past a slit cutter 360 that operates to cut a slit 195 extending in the cross direction CD in the first carrier substrate 202a. As shown in FIGS. 9 and 9A, the slit 195 may be positioned adjacent the leading edge 230 of the elastic part 200. It is to be appreciated that in some configurations, the slit 195 may be adjacent the trailing edge of the elastic part 200. It is further to be appreciated that the slit may also extend through a portion of the elastic part 200. The slit 195 in the first carrier substrate 202a corresponds with the slit 195 described above with reference to the absorbent article 100, and as such, may have various lengths and shapes, and may be positioned relative to other absorbent article components as described above. It is to be appreciated that the slit cutter 360 may be configured in various ways, such as for example, a flex knife, a die knife, a perforator, and a laser.

As shown in FIG. 3, the bonding device 324 may be configured as a mechanical bonding device that includes an anvil roll 350. The anvil roll 350 may include an outer circumferential surface 352 and rotates about an axis of rotation 354, wherein the anvil roll 350 may rotate in a fifth direction Dir5 that is opposite the fourth direction Dir4. The outer circumferential surface 352 of the anvil roll 350 may define the pressing surface 342 operating in conjunction with the pattern roll 340. As shown in FIG. 3, the second carrier substrate 202b may advance in the machine direction and onto the outer circumferential surface 352 of the anvil roll 350. In some configurations, the outer circumferential surface 346 of the pattern roll 340 may also comprise one or more bonding surfaces defined by bonding elements extending radially outward. As the pattern roll 340 and the anvil roll 350 rotate, the first carrier substrate 202a, discrete elastic parts 200, and the second carrier substrate 202b are advanced between the bonding surfaces 356 and the pressing surface 342 to mechanically bond or weld the first and second carrier substrates 202a, 202b and elastic part 200 together to create the pressure bonds 190 discussed above. Heat and/or pressure between the pressing surface 342 and the pattern roll 340 may melt and bond the carrier substrates 202a, 202b and the elastic part 200 together in areas supported by the bonding surfaces on the pattern roll 340. The mechanical bonds and/or bond regions 190 may have shapes that correspond with and may mirror shapes of the bonding surfaces.

As the laminate 204 advances through the nip 344, the carrier substrate 202a, 202b and the discrete elastic part 200 may be mechanically bonded or welded together. It is to be appreciated that the bonding device 324 herein may be configured in various ways with various features described herein to bond the discrete elastic parts 200 with the carrier substrates 202a, 202b. As such, the pattern roll 340 and/or anvil roll 350 may be configured to apply heat and pressure in various ways to perform mechanical bonding, such as for example, the mechanical bonding devices and methods disclosed in in U.S. Pat. Nos. 4,854,984; 6,248,195; 8,778,127; 9,005,392; 9,962,297; and 10,052,237, which are all incorporated by reference herein. It is also to be appreciated that the positions of the pattern roll 340 and anvil roll 350 may be opposite to that which is illustrated in FIG. 3. It is also to be appreciated that one or more components of the bonding device 324 may be configured to operate at constant and/or variable speeds. For example, the pattern roll 340 and/or the anvil roll 350 may be connected with various types of motors, such as servo motors for example, that may rotate the pattern roll 340 and/or the anvil roll 350 at constant and/or variable angular velocities.

In some configurations, the second carrier substrate 202b may be partially wrapped around the outer circumferential surface 352 of the anvil roll 352 upstream of the nip 344. As such, the bonding device 324 may include one or more rolls that help guide the second carrier substrate 202b to the anvil roll 350. For example, as shown in FIG. 3, the bonding device 324 may include a guide roll 361 that helps to guide the second carrier substrate 202b onto the outer circumferential surface 352 of the anvil roll 350.

It is to be appreciated that the bonding device 324 may be configured in various ways, such as with heated or unheated pattern rolls, anvil rolls and/or ultrasonic bonding devices. For example, the bonding device 324 may include the pattern roll 340 and the pressing surface 342 that comprises an energy transfer surface of an ultrasonic bonding device. As such, the bonding device may include a horn and may be configured to impart ultrasonic energy to the combined elastic part 200 and the first carrier substrate 202a on the pattern roll 340. It is to be appreciated that aspects of the ultrasonic bonding device may be configured in various ways, such as for example linear or rotary type configurations, and such as disclosed for example in U.S. Pat. Nos. 3,113,225; 3,562,041; 3,733,238; 5,110,403; 6,036,796; 6,508,641; and 6,645,330, which are all incorporated by reference herein. In some configurations, the ultrasonic bonding device may be configured as a linear oscillating type sonotrode, such as for example, available from Herrmann Ultrasonic, Inc. In some configurations, the sonotrode may include a plurality of sonotrodes nested together in the cross direction CD. It is also to be appreciated that rotary horns may also be configured to rotate at constant and/or variable angular velocities.

As shown in FIG. 3, after the discrete elastic part 200 is bonded with the first and second carrier substrates 202a, 202b to create the laminate 204, the laminate 204 may continue to advance in the machine direction MD from the bonding device 324 and may be subjected to additional converting operations, such as cutting, folding, and/or packaging operations. In some configurations, the laminate 204 may define a continuous length of absorbent articles or may be combined with additional substrates and/or components to define a continuous length of absorbent articles. In turn, the continuous length of absorbent articles may be subjected to a final knife cut that separates discrete absorbent articles from the continuous length of absorbent articles. As previously mentioned, the discrete elastic parts 200 may correspond with waist panels 158 on the absorbent articles 100; the first carrier substrate 202a may correspond with a topsheet substrate 138, and the second carrier substrate 202b may correspond with the backsheet substrate 136. In some configurations, the apparatuses and methods herein may be configured to apply discrete elastic parts 200 as discrete front and/or back waist panels 158. In some configurations, the discrete elastic parts 200 may be applied between the first and second carrier substrates 202a, 202b, and the discrete elastic parts 200 may be subsequently cut during the final knife cut operation into a front waist panel 158a positioned in the front waist region 116 and a back waist panel 158b positioned in the back waist region 118. It is to be appreciated that such final knife cut operation may be configured to apply straight and/or curved cut lines through the carrier substrates 202a, 202b and discrete elastic parts 200. It is also to be appreciated that the carrier substrates 202a, 202b may include parts, such as laterally extending side panels for example, attached thereto upstream of the bonding device 324. As such, the system 300 may also include devices, such as rails and/or conveyors, to help guide and control the carrier substrates 202a, 202b, and specifically such laterally extending features, into the bonding device 324 to help prevent unintentional bonding of such features.

As discussed above with reference to FIG. 3, the system 300 may include a second adhesive applicator device 303 that may be configured to apply second adhesive 222b to the continuous elastic substrate 200a upstream of the nip 310 between the knife roll 306 and anvil roll 308. In turn, the discrete elastic parts 200 separated from the continuous elastic substrate 200a may include a zone 240 of second adhesive 222b that is adapted to adhesively bond the elastic part 200 with the second carrier substrate 202b. It is to be appreciated that the zone 240 of second adhesive 222b may comprise adhesive 222 applied to the continuous elastic substrate 200a, the elastic part 200, and/or the second carrier substrate 202b in various configurations and/or positions in the assembly process. For example, as shown in FIG. 3, the system 300 may include an adhesive applicator device 303a that may be configured to apply second adhesive 222 to the discrete elastic part 200 at a position downstream of the nip 310 between the knife roll 306 and anvil roll 308. In another example, shown in FIG. 3, the apparatus 300 may include an adhesive applicator device 303b that deposits second adhesive 222b onto the first surface 210b of the second carrier substrate 202b to define the zone 240 of adhesive 222b that bonds the elastic part 200 with the second carrier substrate 202b. It is to be appreciated that the adhesive applicator device 303a may be configured to operate in addition to or in place of the adhesive applicators 303, 303b, and adhesive applicator device 303b may be configured to operate in addition to or in place of the adhesive applicators 303, 303a. It is also to be appreciated that the adhesive applicator devices 303a, 303b may be configured in various ways, such as the adhesive applicator 303 described above, such as for example, as a spray nozzle and/or a slot coating device. It is also to be appreciated that in some configurations, the discrete elastic parts 200 may be combined with the carrier substrates 202a, 202b with only mechanical bonds and without the use of adhesive.

It is also to be appreciated that the waist panels 158 herein may be assembled in various ways, such as for example, the continuous elastic substrate and the discrete elastic parts as disclosed in U.S. Pat. Nos. 6,572,595; 6,830,800; 7,087,287; and 7,803,244; U.S. patent application Ser. No. 18/492,918; and U.S. Patent Publication Nos. 2018/0042778 A1; 2018/0042787 A1; 2018/0042779 A1; and 2018/0042780 A1; 2020/0375807 A1; 2020/0375815 A1; 2021/0128366 A1; and 2021/0128369 A1, which are all incorporated by reference herein.

Although the apparatus 300 and associated operations described above with reference FIG. 3 relate to elastic parts 200 cut from an elastic laminate 200a that is configured to stretch in the cross direction CD upstream of the nip 310, it is to be appreciated that the apparatus 300 may be adapted operate to bond discrete elastic parts 200 cut from an elastic laminate 200a adapted to stretch in the machine direction MD upstream of the nip 310. In such a configuration, the transfer device 322 may be configured as a turn and repitch apparatus 370 that turns/rotates and repitches the discrete elastic parts 200, as opposed to the spreader device 326 described above.

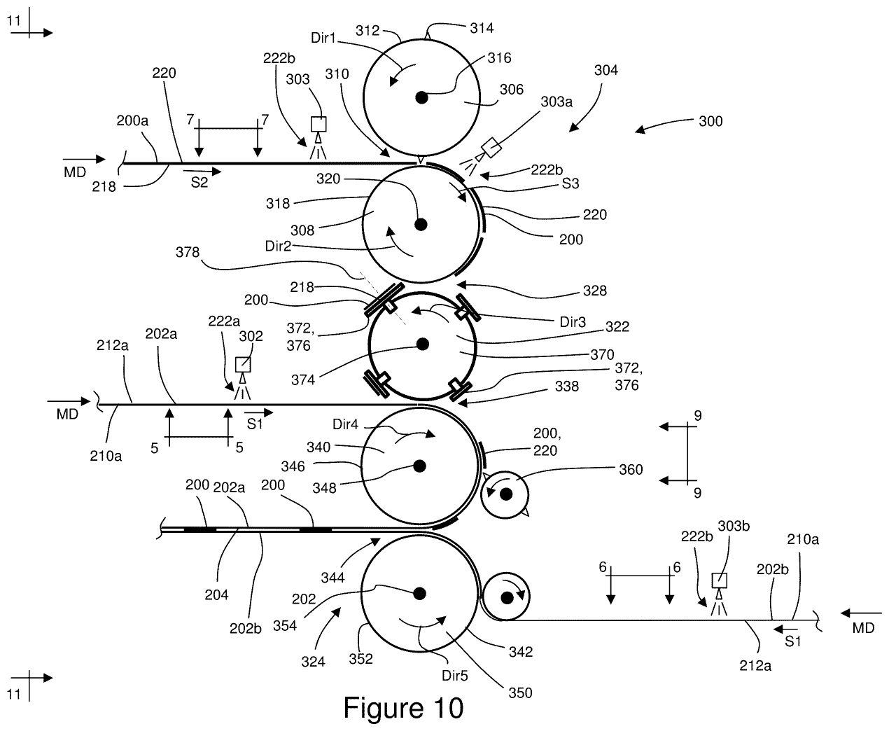

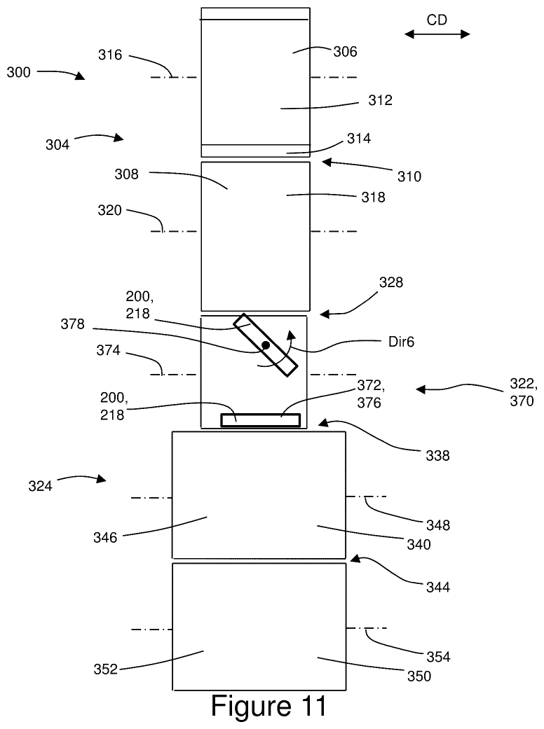

For example, FIGS. 10 and 11 provide schematic representations of an apparatus configured to bond discrete elastic parts 200 cut from an elastic laminate 200a adapted to stretch in the machine direction MD upstream of the nip 310. It is to be appreciated that many of the component parts numbered and shown in FIGS. 10 and 11 are the same as those described above with reference to FIGS. 3 and 4. With reference to FIG. 10, a continuous elastic substrate 200a is advanced at a second speed S2 in a machine direction MD, wherein the continuous elastic substrate 200a is stretchable in the machine direction MD.

As shown in FIGS. 10 and 11, the continuous elastic substrate 200a may advance in the machine direction MD to the cutting device 304 that cuts and separates discrete elastic parts 200 from the continuous elastic substrate 200a. The elastic part 200 includes first and second surfaces 218, 220 that correspond with the first and second surfaces 218, 220 of the continuous elastic substrate 200a. The cutting device 304 may be configured as the cutting device described above with reference to FIGS. 3 and 4. As such, the outer circumferential surface 318 of the anvil roll 308 may advance at a third speed S3. In some configurations, the third speed S3 is greater than the second speed S2, and as such, the continuous elastic substrate 200a is stretched in the machine direction MD.

With continued reference to FIG. 10, as the continuous elastic substrate 200a advances through the nip 310 between the knife roll 306 and the anvil roll 310, the blade 314 operates to cut the discrete elastic part 200 from the continuous elastic substrate 200a. Because the outer circumferential surface 318 of the anvil roll 308 advances at the third speed S3 that is greater than the second speed S2, the cut discrete elastic part 200 is in a stretched state on the outer circumferential surface 318 of the anvil roll 308. The anvil roll 308 may also be configured to apply vacuum pressure to the discrete elastic parts 200 to help hold the discrete elastic parts 200 on the outer circumferential surface 318 in a stretched state as the anvil roll 308 rotates.

With reference to FIG. 10, the apparatus 300 may include a rotatable transfer device 322 that transfers the discrete elastic parts 200 from the cutting device 304 to a bonding device 324, which in turn, combines the elastic parts 200 with the first carrier substrate 202a and the second carrier substrate 202b. As mentioned above, the transfer device 322 may be configured as turn and repitch device. As shown in FIGS. 10 and 11, the transfer device 322 is configured to transport the stretched discrete elastic parts in a machine direction MD from the nip 328 between the anvil roll 308 and the transfer device 322 to the nip 338 between the transfer device 322 and the bonding device 324. However, the transfer device 322 may also be configured to reorient the discrete elastic parts 200 from a first orientation on the anvil roll 308 having the direction of stretch in the machine direction MD to a second orientation onto the second surface 212a of the first carrier substrate 202a with the direction of stretch in the cross direction CD. For example, the turn and repitch apparatus 370 may comprise and one or more transfer members 372 adapted to orbit as indicated by directional arrow Dir3 in a machine direction MD about a first axis 374. In turn, each transfer member 372 may include a carrier surface 376 adapted to engage the discrete elastic parts 200. As shown in FIGS. 10 and 11, the carrier surface 376 may also be rotatable about a second axis 378 as indicated by directional arrow Dir6 between the first orientation and the second orientation. The carrier surface may be configured to be in communication with a vacuum source to help hold the elastic parts 200 on the carrier surfaces 376 in a stretched state. As such, the carrier surface 376 and the elastic part 200 positioned on the carrier surface 376 orbit about the first axis 374 from the nip 328 to the nip 338. While orbiting from the nip 328 to the nip 338, the carrier surface 376 and the discrete elastic part 200 are rotated about the second axis of rotation 378 to place the carrier surface 376 and the elastic part 200 in a second orientation. In particular, in the second orientation, the direction of stretch of the elastic part 200 is perpendicular or substantially perpendicular to the machine direction MD. Once the elastic parts are transferred to the first carrier substrate 202a at the nip 338, the bonding device 324, the apparatus 300 of FIGS. 10 and 11 may operate as described above with reference to FIGS. 3 and 4.

It is to be appreciated that the transfer device 322 shown in FIGS. 10 and 11 may be configured in various ways, for example, such as disclosed in U.S. patent application Ser. No. 18/492,918; U.S. Pat. Nos. 7,587,966; 9,168,182; 9,737,442; 2021/0267812; and U.S. Patent Publication No. 2022/0008256; which are all incorporated by reference herein. It is also to be appreciated that the transfer device 322 may be configured as a combination of a turning device that performs a step of reorienting the elastic parts 200 and a separate device that performs repitching step.

### Combinations

A1. A method for assembling absorbent articles, the method comprising steps of: providing a first carrier substrate; advancing the first carrier substrate in a machine direction; providing an elastic part comprising a first surface and an opposing second surface, the elastic part further comprising a first longitudinal edge and a second longitudinal edge separated from the first longitudinal edge in a cross direction, the elastic part further comprising a first lateral edge and a second lateral edge separated from the first lateral edge in the machine direction; bonding the first surface of the elastic part with the first carrier substrate; cutting a slit in the first carrier substrate, the slit extending in the cross direction; providing a second carrier substrate; sandwiching the elastic part between the first carrier substrate and the second carrier substrate; and bonding a first region of the second surface the elastic part with the second carrier substrate, wherein a second region of the second surface of the elastic part remains unattached to the second carrier substrate to define a pocket between the second region of the second surface of the elastic part and the second carrier substrate, the pocket comprising an opening defined by the slit in the first carrier substrate.

A2. The method of paragraph A1, wherein the first lateral edge comprises a leading edge and the second lateral edge comprises a trailing edge.

A3. The method of paragraph A1 or A2, wherein the first carrier substrate comprises a topsheet substrate.

A4. The method of any of paragraphs A1 to A3, wherein the step of providing the first carrier substrate further comprises positioning an absorbent core on the first carrier substrate before the step of bonding the first surface of the elastic part.

A5. The method of paragraph A4, wherein the slit is positioned upstream or downstream in the machine direction from the absorbent core.

A6. The method of any of paragraphs A1 to A5, wherein the second carrier substrate comprises a backsheet substrate.

A7. The method of paragraph A6, wherein the step of providing the second carrier substrate further comprises positioning an absorbent core on the second carrier substrate before the step of sandwiching the elastic part.

A8. The method of paragraph A7, wherein a portion of the absorbent core is positioned between the second carrier substrate and the slit in the first carrier substrate.

A9. The method of any of paragraphs A1-A8, wherein the step of providing the elastic part further comprises cutting the elastic part from a continuous elastic substrate, wherein the elastic part comprises a first end region and a second end region separated from the first end region by a central region.

A10. The method of paragraph A9, further comprising a step of stretching the central region of the elastic part.

A11. The method of paragraph A10, wherein the first end region is separated from the second end region in the cross direction by the central region, and wherein the step of providing the elastic part further comprises stretching the central region of the elastic part in the cross direction.

A12. The method of paragraph A9, wherein the first end region is separated from the second end region in the machine direction by the central region, and wherein the step of providing the elastic part further comprises at least one of stretching the continuous elastic substrate in the machine direction and stretching the central region of the elastic part in the machine direction.

A13. The method of paragraph A12, further comprising a step of rotating the discrete elastic part such that the second end region is separated from the first end region in the cross direction by the central region.

A14. The method of paragraph A9, further comprising steps of: advancing the first carrier substrate at a first speed; advancing the continuous elastic substrate at a second speed; and changing a speed of the elastic part from the second speed to the first speed.

A15. The method of any of paragraphs A1 to A14, wherein the step of bonding the first surface of the elastic part with the first carrier substrate further comprises applying adhesive to the first surface of the elastic part.

A16. The method of any of paragraphs A1 to A15, wherein the step of bonding the first surface of the elastic part with the first carrier substrate further comprises applying adhesive to the first carrier substrate.

A17. The method of any of paragraphs A1 to A16, wherein the step of bonding the first region of the second surface of the elastic part with the second carrier substrate further comprises applying adhesive to the second surface of the elastic part.

A18. The method of any of paragraphs A1 to A17, wherein the step of bonding the first region of the second surface of the elastic part with the second carrier substrate further comprises applying adhesive to the second carrier substrate.

A19. The method of any of paragraphs A1 to A18, wherein the step of bonding the first surface of the elastic part with the first carrier substrate further comprises applying at least one of ultrasonic bonds, pressure bonds, and thermal bonds.

A20. The method of any of paragraphs A1 to A19, wherein the step of bonding the first region of the second surface of the elastic part with the second carrier substrate further comprises applying at least one of ultrasonic bonds, pressure bonds, and thermal bonds.

A21. The method of any of paragraphs A1 to A20, wherein the slit is positioned adjacent the first lateral edge.

A22. The method of any of paragraphs A1 to A21, wherein the step of cutting the slit in the first carrier substrate further comprises cutting the elastic part.

B1. An absorbent article comprising: a front waist region, a back waist region, and a crotch region disposed between the front and back waist regions; a first waist edge, a second waist edge longitudinally separated from the first waist edge, a first side edge; and a second side edge laterally separated from the first side edge; a chassis comprising a topsheet, a backsheet, and an absorbent core positioned between the topsheet and the backsheet; a slit in the topsheet; a waist panel comprising an inboard lateral edge, an outboard lateral edge, a first longitudinal edge, and a second longitudinal edge, wherein the waist panel further comprises a first surface and an opposing second surface; wherein the first surface of the waist panel is bonded with a garment facing surface of the topsheet, and wherein a first region of the second surface the waist panel is bonded with a wearer facing surface of the backsheet, wherein a second region of the second surface of the waist panel is unattached to the backsheet to define a pocket between the second region of the second surface of the waist panel and the backsheet, the pocket comprising an opening defined by the slit in the topsheet.

B2. The absorbent article of paragraph B1, wherein the first region is positioned longitudinally outboard of the second region.

B3. The absorbent article of paragraph B1 or B2, wherein a portion of the absorbent core is positioned between the slit and the backsheet.

B4. The absorbent article of any of paragraphs B1 to B3, wherein the waist panel comprises at least one of elastic strands and elastic film.

B5. The absorbent article of any of paragraphs B1 to B4, wherein a portion of the inboard lateral edge of the waist panel is unattached to the backsheet.

### Bio-Based Content for Components

Components of the absorbent articles described herein may at least partially be comprised of bio-based content as described in U.S. Pat. Appl. No. 2007/0219521A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for assembling absorbent articles, the method comprising steps of:
providing a first carrier substrate;
advancing the first carrier substrate in a machine direction;
providing an elastic part comprising a first surface and an opposing second surface, the elastic part further comprising a first longitudinal edge and a second longitudinal edge separated from the first longitudinal edge in a cross direction, the elastic part further comprising a first lateral edge and a second lateral edge separated from the first lateral edge in the machine direction;
bonding the first surface of the elastic part with the first carrier substrate;
cutting a slit in the first carrier substrate, the slit extending in the cross direction;
providing a second carrier substrate;
sandwiching the elastic part between the first carrier substrate and the second carrier substrate; and
bonding a first region of the second surface the elastic part with the second carrier substrate, wherein a second region of the second surface of the elastic part remains unattached to the second carrier substrate to define a pocket between the second region of the second surface of the elastic part and the second carrier substrate, the pocket comprising an opening defined by the slit in the first carrier substrate.

2. The method of claim 1, wherein the first lateral edge comprises a leading edge and the second lateral edge comprises a trailing edge.

3. The method of claim 1, wherein the first carrier substrate comprises a topsheet substrate.

4. The method of claim 3, wherein the step of providing the first carrier substrate further comprises positioning an absorbent core on the first carrier substrate before the step of bonding the first surface of the elastic part.

5. The method of claim 4, wherein the slit is positioned upstream or downstream in the machine direction from the absorbent core.

6. The method of claim 1, wherein the second carrier substrate comprises a backsheet substrate.

7. The method of claim 6, wherein the step of providing the second carrier substrate further comprises positioning an absorbent core on the second carrier substrate before the step of sandwiching the elastic part.

8. The method of claim 7, wherein a portion of the absorbent core is positioned between the second carrier substrate and the slit in the first carrier substrate.

9. The method of claim 1, wherein the step of providing the elastic part further comprises cutting the elastic part from a continuous elastic substrate, wherein the elastic part comprises a first end region and a second end region separated from the first end region by a central region.

10. The method of claim 9, further comprising a step of stretching the central region of the elastic part.

11. The method of claim 10, wherein the first end region is separated from the second end region in the cross direction by the central region, and wherein the step of providing the elastic part further comprises stretching the central region of the elastic part in the cross direction.

12. The method of claim 9, wherein the first end region is separated from the second end region in the machine direction by the central region, and wherein the step of providing the elastic part further comprises at least one of stretching the continuous elastic substrate in the machine direction and stretching the central region of the elastic part in the machine direction.

13. The method of claim 12, further comprising a step of rotating the discrete elastic part such that the second end region is separated from the first end region in the cross direction by the central region.

14. The method of claim 9, further comprising steps of:
advancing the first carrier substrate at a first speed;
advancing the continuous elastic substrate at a second speed; and
changing a speed of the elastic part from the second speed to the first speed.

15. The method of claim 1, wherein the step of bonding the first surface of the elastic part with the first carrier substrate further comprises applying adhesive to the first surface of the elastic part.

16. The method of claim 1, wherein the step of bonding the first surface of the elastic part with the first carrier substrate further comprises applying adhesive to the first carrier substrate.

17. The method of claim 1, wherein the step of bonding the first region of the second surface of the elastic part with the second carrier substrate further comprises applying adhesive to the second surface of the elastic part.

18. The method of claim 1, wherein the step of bonding the first region of the second surface of the elastic part with the second carrier substrate further comprises applying adhesive to the second carrier substrate.

19. The method of claim 1, wherein the step of bonding the first surface of the elastic part with the first carrier substrate further comprises applying at least one of ultrasonic bonds, pressure bonds, and thermal bonds.

20. The method of claim 1, wherein the step of bonding the first region of the second surface of the elastic part with the second carrier substrate further comprises applying at least one of ultrasonic bonds, pressure bonds, and thermal bonds.

21. The method of claim 1, wherein the slit is positioned adjacent the first lateral edge.

22. The method of claim 1, wherein the step of cutting the slit in the first carrier substrate further comprises cutting the elastic part.

23. An absorbent article comprising:
a front waist region, a back waist region, and a crotch region disposed between the front and back waist regions;
a first waist edge, a second waist edge longitudinally separated from the first waist edge, a first side edge; and a second side edge laterally separated from the first side edge;
a chassis comprising a topsheet, a backsheet, and an absorbent core positioned between the topsheet and the backsheet;
a slit in the topsheet;
a waist panel comprising an inboard lateral edge, an outboard lateral edge, a first longitudinal edge, and a second longitudinal edge, wherein the waist panel further comprises a first surface and an opposing second surface;
wherein the first surface of the waist panel is bonded with a garment facing surface of the topsheet, and wherein a first region of the second surface the waist panel is bonded with a wearer facing surface of the backsheet, wherein a second region of the second surface of the waist panel is unattached to the backsheet to define a pocket between the second region of the second surface of the waist panel and the backsheet, the pocket comprising an opening defined by the slit in the topsheet.

24. The absorbent article of claim 23, wherein the first region is positioned longitudinally outboard of the second region.

25. The absorbent article of claim 23, wherein a portion of the absorbent core is positioned between the slit and the backsheet.

26. The absorbent article of claim 23, wherein the waist panel comprises at least one of elastic strands and elastic film.

27. The absorbent article of claim 23, wherein a portion of the inboard lateral edge of the waist panel is unattached to the backsheet.
